SEQUENCE LISTING (16 sequences)

SEQ ID NO: 1 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 2 - protein, 186 aa, Homo sapiens
PVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 3 - protein, 116 aa, Homo sapiens
AHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 4 - protein, 117 aa, Homo sapiens
FAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRR

SEQ ID NO: 5 - protein, 1312 aa, Homo sapiens
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 6 - protein, 1337 aa, Homo sapiens
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 7 - protein, 1274 aa, Homo sapiens
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 8 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 9 - DNA, 576 bp, Homo sapiens
atgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 10 - DNA, 624 bp, Homo sapiens
atgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggcttag

SEQ ID NO: 11 - DNA, 1260 bp, Homo sapiens
atgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaa

SEQ ID NO: 12 - DNA, 1065 bp, Homo sapiens
atgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttga

SEQ ID NO: 13 - DNA, 4017 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctctatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctag

SEQ ID NO: 14 - DNA, 4071 bp, Homo sapiens
atgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaa

SEQ ID NO: 15 - DNA, 3897 bp, Homo sapiens
atgcagcggggcgccgcgctgtgcctgcgactgtggctctgcctgggactcctggacggcctggtgagtgactactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctcaactgcaccgtgtgggctgagtttaactcaggtgtcacctttgactgggactacccagggaagcaggcagagcggggtaagtgggtgcccgagcgacgctcccaacagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggaccgtatctgtgcagcgtgtgcagacccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcagcgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcgcgtgagagacggcacaaggatgagggccccggagctggccactcccgccatacgccacatcatgctgaactgctggtccggagaccccaaggcgagacctgcattctcggacctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtag

SEQ ID NO: 16 - DNA, 2481 bp, Homo sapiens
atggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactga

(19) United States
(12) Patent Application Publication
(10) Pub. No.: US 2011/0200612 A1
(43) Pub. Date: Aug. 18, 2011

(54) TREATMENT OF EYE DISEASES AND EXCESSIVE NEOVASCULARIZATION USING COMBINED THERAPY

(76) Inventors: Michael Schuster, New York, NY (US); Silviu Itescu, Melbourne, Victoria (AU)

(21) Appl. No.: 13/002,229

(22) PCT Filed: Jun. 29, 2009
(86) PCT No.: PCT/US2009/003902

§ 371 Date: Apr. 27, 2011

Related U.S. Application Data
(60) Provisional application No. 61/133,607, filed on Jun. 30, 2008.

(30) Foreign Application Priority Data
Jun. 30, 2008 (AU) 2008903349

(51) Int. Cl. A61K 39/395; A61K 35/00; A61K 35/12; A61K 35/44; A61K 35/28; A61P 27/02; A61P 9/00

(52) U.S. Cl. 424/158.1; 424/93.1; 424/93.7; 424/93.2

(57) ABSTRACT

The present invention relates to methods of treating or preventing eye diseases, as well as angiogenesis-related diseases, by combination therapy involving administration of cells and a compound that disrupts VEGF-signalling.

### FIELD OF THE INVENTION

The present invention relates to methods of treating or preventing eye diseases, as well as angiogenesis-related diseases, by a combination therapy involving the administration of cells and a compound that disrupts VEGF-signalling.

### BACKGROUND OF THE INVENTION

### Angiogenesis

Angiogenesis (or neovascularisation) is the formation and differentiation of new blood vessels. Angiogenesis is generally absent in healthy adult or mature tissue. However, it occurs in the healthy body for healing wounds and for restoring blood flow to tissues after injury or insult. In females, angiogenesis also occurs during the monthly reproductive cycle and during pregnancy. Under these processes, the formation of new blood vessels is strictly regulated.

### Angiogenesis and Disease

In many serious disease states, the body loses control over angiogenesis. Excessive angiogenesis occurs in diseases such as cancer, macular degeneration, diabetic retinopathy, arthritis, and psoriasis. In these conditions, new blood vessels feed diseased tissues, destroy normal tissues, and in the case of cancer, the new vessels allow tumor cells to escape into the circulation and lodge in other organs (tumor metastasis).

The hypothesis that tumor growth is angiogenesis-dependent was first proposed in 1971 (Folkman, 1971). In its simplest terms the hypothesis proposes that expansion of tumor volume beyond a certain phase requires the induction of new capillary blood vessels. For example, pulmonary micrometastases in the early prevascular phase in mice would be undetectable except by high power microscopy on histological sections. Further indirect evidence supporting the concept that tumor growth is angiogenesis dependent is found in U.S. Pat. Nos. 5,639,725, 5,629,327, 5,792,845, 5,733,876, and 5,854,205.

To stimulate angiogenesis, tumors upregulate their production of a variety of angiogenic factors, including the fibroblast growth factors (αFGF and βFGF) (Kandel et al., 1991) and vascular endothelial cell growth factor/vascular permeability factor (VEGF/VPF) and HGF. However, many malignant tumors also generate inhibitors of angiogenesis, including angiostatin protein and thrombospondin. (Chen et al., 1995; Good et al., 1990; O'Reilly et al., 1994). It is postulated that the angiogenic phenotype is the result of a net balance between these positive and negative regulators of neovascularization. (Good et al., 1990; O'Reilly et al., 1994). Several other endogenous inhibitors of angiogenesis have been identified, although not all are associated with the presence of a tumor. These include, platelet factor 4 (Gupta et al., 1995; Maione et al., 1990), interferon-alpha, interferon-inducible protein 10 (Angiolillo et al., 1995; Strieter et al., 1995), which is induced by interleukin-12 and/or interferon-gamma (Voest et al., 1995), gro-beta (Cao et al., 1995), and the 16 kDa N-terminal fragment of prolactin (Clapp et al., 1993).

### Eye Diseases

A number of eye diseases or disorders caused by dysfunction of tissues or structures in the eye may lead to diminished visual acuity or total loss of vision. Ophthalmic diseases have increased recently, including diseases such as dry eye and asthenopia due to wide use of television, computers, game machines and other digital appliances, and contact lenses.

Of the ocular diseases, age-related macular degeneration (AMD) is particularly prevalent among the aged population of Western society. AMD is the most common cause of legal, irreversible blindness in patients aged 65 and over in the US, Canada, England, Wales, Scotland and Australia. Although the average age of patients when they lose central vision in their first eye is about 65 years, some patients develop evidence of the disease in their fourth or fifth decade of life. The number of people afflicted by this disease is steadily increasing owing to our modern lifestyle and increasing life expectancy.

Neovascularization in the eye is the basis of severe ocular diseases such as AMD and Diabetic retinopathy. Approximately 10% to 15% of patients manifest the exudative (wet) form of the disease. Exudative AMD is characterized by angiogenesis and the formation of pathological neovasculature. The disease is bilateral with accumulating chances of approximately 10% to 15% per annum of developing the blinding disorder in the fellow eye.

Diabetic retinopathy is a complication of diabetes that occurs in approximately 40 to 45 percent of those diagnosed with either Type I or Type II diabetes. Diabetic retinopathy usually effects both eyes and progresses over four stages. The first stage, mild nonproliferative retinopathy, is characterized by microaneuryisms in the eye. Small areas of swelling in the capillaries and small blood vessels of the retina occurs. In the second stage, moderate nonproliferative retinopathy, the blood vessels that supply the retina become blocked. In severe nonproliferative retinopathy, the third stage, the obstructed blood vessels lead to a decrease in the blood supply to the retina, and the retina signals the eye to develop new blood vessels (angiogenesis) to provide the retina with blood supply. In the fourth and most advanced stage, proliferative retinopathy, angiogenesis occurs, but the new blood vessels are abnormal and fragile and grow along the surface of the retina and vitreous gel that fills the eye. When these thin blood vessels rupture or leak blood, severe vision loss or blindness can result.

Bevacizumab is a compound which has been used to treat AMD, however, a side-effect of this therapy is an increase in retinal detachment (Chan et al., 2007; Kook et al., 2008; Garg et al., 2008).

With age, the vitreous humor changes from gel to liquid and as it does so it gradually shrinks and separates from the ILM of the retina. This process is known as “posterior vitreous detachment” (PVD) and is a normal occurrence after age 40. However, degenerative changes in the vitreous may also be induced by pathological conditions such as diabetes, Eale's disease and uveitis. Also, PVD may occur earlier than normal in nearsighted people and in those who have had cataract surgery. Usually, the vitreous makes a clean break from the retina. Occasionally, however, the vitreous adheres tightly to the retina in certain places. These small foci of resisting, abnormally firm attachments of the vitreous can transmit great tractional forces from the vitreous to the retina at the attachment site. This persistent tugging by the vitreous often results in horseshoe-shaped tears in the retina. Unless the retinal tears are repaired, vitreous fluid can seep through this tear into or underneath the retina and cause a retinal detachment, a very serious, sight-threatening condition. In addition, persistent attachment between the vitreous and the ILM can result in bleeding from rupture of blood vessels, which results in the clouding and opacification of the vitreous.

The development of an incomplete PVD has an impact on many vitreoretinal diseases including vitreomacular traction syndrome, vitreous hemorrhage, macular holes, macular edema, diabetic retinopathy, diabetic maculopathy and retinal detachment. There is a need for additional therapies that can be used to treat or prevent eye diseases and/or angiogenesis-related disorders.

### SUMMARY OF THE INVENTION

The present inventors have surprisingly found that a combination therapy comprising cells and a compound that disrupts VEGF-signalling is synergistic when used to treat or prevent eye diseases. Thus, in a first aspect, the present invention provides a method of treating or preventing an eye disease in a subject, the method comprising administering to the subject i) cells, and ii) a compound that disrupts vascular endothelial growth factor (VEGF)-signalling.

Examples of eye diseases which can be treated or prevented using the methods of the invention include, but are not limited to, retinal ischemia, retinal inflammation, retinal edema, retinal detachment, macular hole, tractional retinopathy, vitreous hemorrhage, tractional maculopathy, diabetic retinopathy, diabetic macular edema, retinopathy of prematurity, macular degeneration, corneal graft rejection, neovascular glaucoma, retrolental fibroplasia and/or rubeosis. In a preferred embodiment, the eye disease is retinal detachment, diabetic retinopathy, retinopathy of prematurity and/or macular degeneration.

In an embodiment, the macular degeneration is dry age-related macular degeneration or wet age-related macular degeneration. Preferably, the macular degeneration is wet age-related macular degeneration.

Previously, the present Applicant has shown that stem cells, or progeny thereof, can be used to treat or prevent angiogenesis-related disorders (see WO 2008/006168). They have also surprisingly found that a combination therapy comprising cells and a compound that disrupts VEGF-signalling is synergistic when used to treat or prevent angiogenesis-related disorders. Thus, in a second aspect, the present invention provides a method of treating or preventing an angiogenesis-related disease in a subject, the method comprising administering to the subject i) cells, and ii) a compound that disrupts vascular endothelial growth factor (VEGF)-signalling.

Examples of angiogenesis-related diseases which can be treated or prevented using the methods of the invention include, but are not limited to, angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease and Helicobacter pylori ulcers.

In an embodiment, the cells are stem cells, or progeny cells thereof. In a preferred embodiment, the stem cells are obtained from bone marrow or the eye.

Preferably, the stem cells are mesenchymal precursor cells (MPC). Preferably, the mesenchymal precursor cells are TNAP⁺, STRO-1⁺, VCAM-1⁺, THY-1⁺, STRO-2⁺, CD45⁺, CD146⁺, 3G5⁺ or any combination thereof. In another embodiment, at least some of the STRO-1⁺ cells are STRO-1^{bri}.

In a further embodiment, the MPCs have not been culture expanded and are TNAP⁺.

In a preferred embodiment, the progeny cells are obtained by culturing MPCs in vitro.

In an embodiment, the compound binds, and/or reduces the production of, a vascular endothelial growth factor. Preferably, the vascular endothelial growth factor is VEGF-A, VEGF-B, VEGF-C and/or VEGF-D. More preferably, the vascular endothelial growth factor is VEGF-A.

In an embodiment, the compound that reduces the production of a vascular endothelial growth factor binds, and/or reduces the production of, hypoxia-inducible factor 1 (HIF-1).

In an alternate embodiment, the compound binds, and/or reduces the production of, a vascular endothelial growth factor receptor. Preferably, the vascular endothelial growth factor receptor is selected from VEGFR1, VEGFR2 and/or VEGFR3. More preferably, the vascular endothelial growth factor receptor is VEGFR1 and/or VEGFR2.

In yet another alternate embodiment, the compound binds, and/or reduces the production of, a molecule involved in intracellular signalling induced by a vascular endothelial growth factor binding a vascular endothelial growth factor receptor such as a VEGFR tyrosine kinase.

In an embodiment, the compound is a polypeptide. More preferably, the polypeptide is an antibody, antibody-related molecule, and/or fragment of any one thereof.

In another embodiment, the compound is a polynucleotide. Examples include, but are not limited to, an antisense polynucleotide, a sense polynucleotide, a catalytic polynucleotide, a duplex RNA molecule, or a polynucleotide encoding any one or more thereof.

In an embodiment, at least some of the cells are genetically modified.

Also provided is the use of cells and a compound that disrupts VEGF-signalling for the manufacture of a medicament(s) for use in a combined therapy for treating or preventing an eye disease in a subject.

Further, provided is the use of cells and a compound that disrupts VEGF-signalling as medicaments for use in a combined therapy for treating or preventing an eye disease in a subject.

Also provided is the use of cells and a compound that disrupts VEGF-signalling for the manufacture of a medicament(s) for use in a combined therapy for treating or preventing an angiogenesis-related disorder in a subject.

Further, provided is the use of cells and a compound that disrupts VEGF-signalling as medicaments for use in a combined therapy for treating or preventing an angiogenesis-related disorder in a subject.

In a further aspect, the present invention provides a composition comprising cells and a compound that disrupts VEGF-signalling, and optionally a pharmaceutically-acceptable carrier.

In another aspect, the present invention provides a kit comprising cells and a compound that disrupts VEGF-signalling. The cells and the compound may be in the same or different containers.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE ACCOMPANYING DRAWINGS

FIG. 1. Study design.

FIG. 2. Allogeneic MPCs are equivalent to, and synergistic with, anti-VEGF, in reducing vascular leakage.

FIG. 3. Combining allogeneic MPCs with anti-VEGF eliminates severely leaky vessels.

FIG. 4. Synergistic benefit of combining allogeneic MPCs and anti-VEGF on high-grade leaky vessels.

FIG. 5. Sustained prevention of Stage 4 disease by combination of allogeneic MPCs and anti-VEGF combination, but only short-lived effect by anti-VEGF alone.

FIG. 6. Combining allogeneic MPCs with anti-VEGF maintains higher proportion of laser-damaged vessels in Stage 1 disease.

FIG. 7. Combining allogeneic MPCs with anti-VEGF prevents retinal detachment.

FIG. 8. Combining allogeneic MPCs with anti-VEGF prevents retinal detachment after laser-induced neovascularization.

### KEY TO SEQUENCE LISTING

SEQ ID NO: 1—Human VEGF-A (active processed peptide).
SEQ ID NO: 2—Human VEGF-B (active processed peptide).
SEQ ID NO: 3—Human VEGF-C (active processed peptide).
SEQ ID NO: 4—Human VEGF-D (active processed peptide).
SEQ ID NO: 5—Human VEGFR-1 (minus signal sequence).
SEQ ID NO: 6—Human VEGFR-2 (minus signal sequence).
SEQ ID NO: 7—Human VEGFR-3 (minus signal sequence).
SEQ ID NO: 8—Human HIF-1α.
SEQ ID NO: 9—Coding sequence for full-length human VEGF-A.
SEQ ID NO: 10—Coding sequence for full-length human VEGF-B.
SEQ ID NO: 11—Coding sequence for full-length human VEGF-C.
SEQ ID NO: 12—Coding sequence for full-length human VEGF-D.
SEQ ID NO: 13—Coding sequence for full-length human VEGFR-1.
SEQ ID NO: 14—Coding sequence for full-length human VEGFR-2.
SEQ ID NO: 15—Coding sequence for full-length human VEGFR-3.
SEQ ID NO: 16—Coding sequence for human HIF-1α.

### DETAILED DESCRIPTION OF THE INVENTION

### General Techniques

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### Treatment or Prevention Diseases

As used herein, the term “subject” (also referred to herein as a “patient”) includes warm-blooded animals, preferably mammals, including humans. The subject may be, for example, livestock (e.g. sheep, cow, horse, donkey, pig), companion animal (e.g. dogs, cats), laboratory test animal (e.g. mice, rabbits, rats, guinea pigs, hamsters), or captive wild animal (e.g. fox, deer). In a preferred embodiment, the subject is a primate. In an even more preferred embodiment, the subject is a human.

As used herein the terms “treating”, “treat” or “treatment” include administering a therapeutically effective amount of cells as defined herein, and a therapeutically effective amount of a compound as defined herein, sufficient to reduce or eliminate at least one symptom of an eye disease and/or an angiogenesis-related disorder. In an embodiment, the disease is wet age-related macular degeneration and the method reduces the severity of the disease and/or delays or prevents the recurrence of the disease. In another embodiment, the method of the invention has an increased length of effect than the administration of a compound that disrupts vascular endothelial growth factor (VEGF)-signalling alone.

As used herein the terms “preventing”, “prevent” or “prevention” include administering a therapeutically effective amount of cells as defined herein, and a therapeutically effective amount of a compound as defined herein, sufficient to stop or hinder the development of at least one symptom of an eye disease and/or an angiogenesis-related disorder.

### Eye Diseases

As used herein, an “eye disease” is a disease, ailment or condition which affects or involves the eye or one of the parts or regions of the eye. The eye includes the eyeball and the tissues and fluids which constitute the eyeball, the periocular muscles (such as the oblique and rectus muscles) and the portion of the optic nerve which is within or adjacent to the eyeball.

In an embodiment, the eye disease is characterized, at least in part, by retinal detachment and/or vascular leakage.

It is to be understood that the method of the present invention may be used to prevent or treat any disease of the eye or associated with the eye, or in an embodiment, any ophthalmic disorder. Examples of eye diseases which can be treated or prevented using the methods of the invention include, but are not limited to, episcleritis, scleritis, diabetic retinopathy, glaucoma, macular degeneration, retinal detachment, achromatopsia/Maskun, amblyopia, anisometropia, Argyll Robertson pupil, astigmatism, anisometropia, blindness, chalazion, color blindness, achromatopsia/Maskun, esotropia, exotropia, floaters, vitreous detachment, Fuchs' dystrophy, hypermetropia, hyperopia, hypertensive retinopathy, iritis, keratoconus, Leber's congenital amaurosis, Leber's hereditary optic neuropathy, macular edema, myopia, nyctalopia, opthalmoplegia, including progressive external opthalmoplegia and internal opthalmoplegia, opthalmoparesis, presbyopia, pterygium, red eye (medicine), retinitis pigmentosa, retinopathy of prematurity, retinoschisis, river blindness, opthalmoplegia, scotoma, snow blindness/arc eye, eyelid disorders, ptosis, extraocular tumours, strabismus.

In one preferred embodiment, the methods of the present invention may be used to prevent or treat macular degeneration. In one embodiment, macular degeneration is characterized by damage to or breakdown of the macula, which in one embodiment, is a small area at the back of the eye. In one embodiment, macular degeneration causes a progressive loss of central sight, but not complete blindness. In one embodiment, macular degeneration is of the dry type, while in another embodiment, it is of the wet type. In one embodiment, the dry type is characterized by the thinning and loss of function of the macula tissue. In one embodiment, the wet type is characterized by the growth of abnormal blood vessels behind the macula. In one embodiment, the abnormal blood vessels hemorrhage or leak, resulting in the formation of scar tissue if untreated. In some embodiments, the dry type of macular degeneration can turn into the wet type. In one embodiment, macular degeneration is age-related, which in one embodiment is caused by an ingrowth of chorioidal capillaries through defects in Bruch's membrane with proliferation of fibrovascular tissue beneath the retinal pigment epithelium.

In another preferred embodiment, the methods of the present invention may be used to prevent or treat retinopathy. In one embodiment, retinopathy refers to a disease of the retina, which in one embodiment is characterized by inflammation and in another embodiment, is due to blood vessel damage inside the eye. In one embodiment, retinopathy is diabetic retinopathy which, in one embodiment, is a complication of diabetes that is caused by changes in the blood vessels of the retina. In one embodiment, blood vessels in the retina leak blood and/or grow fragile, brush-like branches and scar tissue, which in one embodiment, blurs or distorts the images that the retina sends to the brain. In another embodiment, retinopathy is proliferative retinopathy, which in one embodiment, is characterized by the growth of new, abnormal blood vessels on the surface of the retina (neovascularization). In one embodiment, neovascularization around the pupil increases pressure within the eye, which in one embodiment, leads to glaucoma. In another embodiment, neovascularization leads to new blood vessels with weaker walls that break and bleed, or cause scar tissue to grow, which in one embodiment, pulls the retina away from the back of the eye (retinal detachment). In one embodiment, the pathogenesis of retinopathy is related to non-enzymatic glycation, glycoxidation, accumulation of advanced glycation end-products, free radical-mediated protein damage, up-regulation of matrix metalloproteinases, elaboration of growth factors, secretion of adhesion molecules in the vascular endothelium, or a combination thereof.

In another preferred embodiment, retinopathy refers to retinopathy of prematurity (ROP), which in one embodiment, occurs in premature babies when abnormal blood vessels and scar tissue grow over the retina. In one embodiment, retinopathy of prematurity is caused by a therapy necessary to promote the survival of a premature infant.

In another preferred embodiment, the methods of the present invention may be used to prevent or treat retinal detachment, including, inter alia, rhegmatogenous, tractional, or exudative retinal detachment, which in one embodiment, is the separation of the retina from its supporting layers. In one embodiment, retinal detachment is associated with a tear or hole in the retina through which the internal fluids of the eye may leak. In one embodiment, retinal detachment is caused by trauma, the aging process, severe diabetes, an inflammatory disorder, neovascularization, or retinopathy of prematurity, while in another embodiment, it occurs spontaneously. In one embodiment, bleeding from small retinal blood vessels may cloud the vitreous during a detachment, which in one embodiment, may cause blurred and distorted images. In one embodiment, a retinal detachment can cause severe vision loss, including blindness.

### Angiogenesis

As used herein, the term “angiogenesis” is defined as a process of tissue vascularization that involves the growth of new and/or developing blood vessels into a tissue, and is also referred to as neo-vascularization. The process can proceed in one of three ways: the vessels can sprout from pre-existing vessels, de novo development of vessels can arise from precursor cells (vasculogenesis), and/or existing small vessels can enlarge in diameter.

As used herein, an “angiogenesis-related disease” is any condition characterized by excessive and/or abnormal neo-vascularization. Any angiogenesis-related disease may be treated or prevented using the methods of the present invention. Angiogenesis-related diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, diabetic macular edema, retinopathy of prematurity, macular degeneration including dry age-related macular degeneration and wet age-related macular degeneration, corneal graft rejection, neovascular glaucoma, retrolental fibroplasia, rubeosis; Osler-Webber Syndrome; myocardial angiogenesis blindness; plaque neovascularization; telangiectasia; hemophiliac joints; angiofibroma; and wound granulation. The methods of the invention are also useful in the treatment or prevention of diseases that have angiogenesis as a pathologic consequence such as cat scratch disease (Rochele minalia quintosa) and ulcers (Helicobacter pylorii).

In a preferred embodiment, the angiogenesis-related disease is an ocular angiogenesis disease. As used herein, an “ocular angiogenesis disease” is any eye disease characterized by excessive and/or abnormal neo-vascularization. Examples include, but are not limited to, diabetic retinopathy, diabetic macular edema, retinopathy of prematurity, macular degeneration, corneal graft rejection, neovascular glaucoma, retrolental fibroplasia and rubeosis.

### Stem Cells and Progeny Thereof

The cell can be any cell type which can be used to treat an eye disease and/or angiogenesis-related disorder.

As used herein, the term “stem cell” refers to self-renewing cells that are capable of giving rise to phenotypically and genotypically identical daughters as well as at least one other final cell type (e.g., terminally differentiated cells). The term “stem cells” includes totipotential, pluripotential and multipotential cells, as well as progenitor and/or precursor cells derived from the differentiation thereof.

As used herein, the term “totipotent cell” or “totipotential cell” refers to a cell that is able to form a complete embryo (e.g., a blastocyst).

As used herein, the term “pluripotent cell” or “pluripotential cell” refers to a cell that has complete differentiation versatility, i.e., the capacity to grow into any of the mammalian body's approximately 260 cell types. A pluripotent cell can be self-renewing, and can remain dormant or quiescent within a tissue.

By “multipotential cell” or “multipotent cell” we mean a cell which is capable of giving rise to any of several mature cell types. As used herein, this phrase encompasses adult or embryonic stem cells and progenitor cells, such as mesenchymal precursor cells (MPC) and multipotential progeny of these cells. Unlike a pluripotent cell, a multipotent cell does not have the capacity to form all of the cell types.

As used herein, the term “progenitor cell” refers to a cell that is committed to differentiate into a specific type of cell or to form a specific type of tissue.

Mesenchymal precursor cells (MPCs) are cells found in bone marrow, blood, dental pulp cells, adipose tissue, skin, spleen, pancreas, brain, kidney, liver, heart, eye including the retina, brain, hair follicles, intestine, lung, lymph node, thymus, bone, ligament, tendon, skeletal muscle, dermis, and periosteum; and are capable of differentiating into different germ lines such as mesoderm, endoderm and ectoderm. Thus, MPCs are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues. Mesenchymal precursor cells are thus non-hematopoietic progenitor cells which divide to yield daughter cells that are either stem cells or are precursor cells which in time will irreversibly differentiate to yield a phenotypic cell.

In a preferred embodiment, cells used in the methods of the invention are enriched from a sample obtained from a subject. The terms ‘enriched’, ‘enrichment’ or variations thereof are used herein to describe a population of cells in which the proportion of one particular cell type or the proportion of a number of particular cell types is increased when compared with the untreated population.

In a preferred embodiment, the cells used in the present invention are TNAP⁺, STRO-1⁺, VCAM-1⁺, THY-1⁺, STRO-2⁺, CD45⁺, CD146⁺, 3G5⁺ or any combination thereof. Preferably, the STRO-1⁺ cells are STRO-1^{bright}. Preferably, the STRO-1^{bright} cells are additionally one or more of VCAM-1⁺, THY-1⁺, STRO-2⁺ and/or CD146⁺.

In one embodiment, the mesenchymal precursor cells are perivascular mesenchymal precursor cells as defined in WO 2004/85630.

When we refer to a cell as being “positive” for a given marker it may be either a low (lo or dim) or a high (bright, bri) expresser of that marker depending on the degree to which the marker is present on the cell surface, where the terms relate to intensity of fluorescence or other colour used in the colour sorting process of the cells. The distinction of lo (or dim or dull) and bri will be understood in the context of the marker used on a particular cell population being sorted. When we refer herein to a cell as being “negative” for a given marker, it does not mean that the marker is not expressed at all by that cell. It means that the marker is expressed at a relatively very low level by that cell, and that it generates a very low signal when detectably labelled.

The term “bright”, when used herein, refers to a marker on a cell surface that generates a relatively high signal when detectably labelled. Whilst not wishing to be limited by theory, it is proposed that “bright” cells express more of the target marker protein (for example the antigen recognised by STRO-1) than other cells in the sample. For instance, STRO-1^{bri} cells produce a greater fluorescent signal, when labelled with a FITC-conjugated STRO-1 antibody as determined by FACS analysis, than non-bright cells (STRO-1^{dull/dim}). Preferably, “bright” cells constitute at least about 0.1% of the most brightly labelled bone marrow mononuclear cells contained in the starting sample. In other embodiments, “bright” cells constitute at least about 0.1%, at least about 0.5%, at least about 1%, at least about 1.5%, or at least about 2%, of the most brightly labelled bone marrow mononuclear cells contained in the starting sample. In a preferred embodiment, STRO-1^{bright} cells have 2 log magnitude higher expression of STRO-1 surface expression. This is calculated relative to “background”, namely cells that are STRO-1⁻. By comparison, STRO-1^{dim} and/or STRO-1^{intermediate} cells have less than 2 log magnitude higher expression of STRO-1 surface expression, typically about 1 log or less than “background”.

When used herein the term “TNAP” is intended to encompass all isoforms of tissue non-specific alkaline phosphatase. For example, the term encompasses the liver isoform (LAP), the bone isoform (BAP) and the kidney isoform (KAP). In a preferred embodiment, the TNAP is BAP. In a particularly preferred embodiment, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in a preferred embodiment, the cells are capable of giving rise to clonogenic CFU-F.

It is preferred that a significant proportion of the multipotential cells are capable of differentiation into at least two different germ lines. Non-limiting examples of the lineages to which the multipotential cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an embodiment, the stem cells, and progeny thereof, are capable of differentiation to pericytes.

In another embodiment, the “multipotential cells” are not capable of giving rise, upon culturing, to hematopoietic cells.

Stem cells useful for the methods of the invention may be derived from adult tissue, an embryo, or a fetus. The term “adult” is used in its broadest sense to include a postnatal subject. In a preferred embodiment, the term “adult” refers to a subject that is postpubertal. The term, “adult” as used herein can also include cord blood taken from a female.

The present invention also relates to use of progeny cells (which can also be referred to as expanded cells) which are produced from the in vitro culture of the stem cells described herein, and include direct progeny of the stem cells as well as progeny thereof and so on. Expanded cells of the invention may have a wide variety of phenotypes depending on the culture conditions (including the number and/or type of stimulatory factors in the culture medium), the number of passages and the like. In certain embodiments, the progeny cells are obtained after about 2, about 3, about 4, about 5, about 6, about 7, about 8, about 9, or about 10 passages from the parental population. However, the progeny cells may be obtained after any number of passages from the parental population.

The progeny cells may be obtained by culturing in any suitable medium. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. The term “medium” also refers to material that is intended for use in a cell culture, even if it has not yet been contacted with cells. In other words, a nutrient rich liquid prepared for bacterial culture is a medium. Similarly, a powder mixture that when mixed with water or other liquid becomes suitable for cell culture, may be termed a “powdered medium”.

In an embodiment, progeny cells useful for the methods of the invention are obtained by isolating TNAP+ cells from bone marrow using magnetic beads labelled with the STRO-3 antibody, and plated in α-MEM supplemented with 20% fetal calf serum, 2 mM L-glutamine and 100 μm L-ascorbate-2-phosphate as previously described (see Gronthos et al. (1995) for further details regarding culturing conditions).

In one embodiment, such expanded cells (at least after 5 passages) can be TNAP−, CC9⁺, HLA class I⁺, HLA class II⁻, CD14⁻, CD19⁻, CD3⁻, CD11a-c⁻, CD31⁻, CD86⁻ and/or CD80⁻. However, it is possible that under different culturing conditions to those described herein that the expression of different markers may vary. Also, whilst cells of these phenotypes may predominate in the expended cell population it does not mean that there is not a minor proportion of the cells that do not have this phenotype(s) (for example, a small percentage of the expanded cells may be CC9−). In one preferred embodiment, expanded cells of the invention still have the capacity to differentiate into different cell types.

In one embodiment, an expended cell population used in the methods of the invention comprises cells wherein at least 25%, more preferably at least 50%, of the cells are CC9⁺.

In another embodiment, an expended cell population used in the methods of the invention comprises cells wherein at least 40%, more preferably at least 45%, of the cells are STRO-1⁺.

In a further embodiment, the progeny cells may express markers selected from the group consisting of LFA-3, THY-1, VCAM-1, ICAM-1, PECAM-1, P-selectin, L-selectin, 3G5, CD49a/CD49b/CD29, CD49c/CD29, CD49d/CD29, CD29, CD18, CD61, integrin beta, 6-19, thrombomodulin, CD10, CD13, SCF, PDGF-R, EGF-R, IGF1-R, NGF-R, FGF-R, Leptin-R, (STRO-2=Leptin-R), RANKL, STRO-1^{bright} and CD146 or any combination of these markers.

In one embodiment, the progeny cells are Multipotential Expanded MPC Progeny (MEMPs) as defined in WO 2006/032092. Methods for preparing enriched populations of MPC from which progeny may be derived are described in WO 01/04268 and WO 2004/085630. In an in vitro context MPCs will rarely be present as an absolutely pure preparation and will generally be present with other cells that are tissue specific committed cells (TSCCs). WO 01/04268 refers to harvesting such cells from bone marrow at purity levels of about 0.1% to 90%. The population comprising MPC from which progeny are derived may be directly harvested from a tissue source, or alternatively it may be a population that has already been expanded ex vivo.

For example, the progeny may be obtained from a harvested, unexpanded, population of substantially purified MPC, comprising at least about 0.1, 1, 5, 10, 20, 30, 40, 50, 60, 70, 80 or 95% of total cells of the population in which they are present. This level may be achieved, for example, by selecting for cells that are positive for at least one marker selected from the group consisting of ‘TNAP, STRO-1^{bright}, 3G5⁺, VCAM-1, THY-1, CD146 and STRO-2.

The MPC starting population may be derived, for example, from any one or more tissue types set out in WO 01/04268 or WO 2004/085630, namely bone marrow, dental pulp cells, adipose tissue and skin, or perhaps more broadly from adipose tissue, teeth, dental pulp, skin, liver, kidney, heart, retina, brain, hair follicles, intestine, lung, spleen, lymph node, thymus, pancreas, bone, ligament, bone marrow, tendon and skeletal muscle.

MEMPS can be distinguished from freshly harvested MPCs in that they are positive for the marker STRO-1^{bri} and negative for the marker Alkaline phosphatase (ALP). In contrast, freshly isolated MPCs are positive for both STRO-1^{bri} and ALP. In a preferred embodiment of the present invention, at least 15%, 20%, 30%, 40%, 50%, 60%, 70%, 80%, 90% or 95% of the administered cells have the phenotype STRO-1^{bri}, ALP⁻. In a further preferred embodiment the MEMPS are positive for one or more of the markers Ki67, CD44 and/or CD49c/CD29, VLA-3, α3β1. In yet a further preferred embodiment the MEMPs do not exhibit TERT activity and/or are negative for the marker CD18.

In one embodiment, the cells are taken from a patient with an angiogenesis related disease, cultured in vitro using standard techniques and administered to a patient as an autologous or allogeneic transplant. In an alternative embodiment, cells of one or more of the established human cell lines are used. In another useful embodiment of the invention, cells of a non-human animal (or if the patient is not a human, from another species) are used.

The invention can be practised using cells from any non-human animal species, including but not limited to non-human primate cells, ungulate, canine, feline, lagomorph, rodent, avian, and fish cells. Primate cells with which the invention may be performed include but are not limited to cells of chimpanzees, baboons, cynomolgus monkeys, and any other New or Old World monkeys. Ungulate cells with which the invention may be performed include but are not limited to cells of bovines, porcines, ovines, caprines, equines, buffalo and bison. Rodent cells with which the invention may be performed include but are not limited to mouse, rat, guinea pig, hamster and gerbil cells. Examples of lagomorph species with which the invention may be performed include domesticated rabbits, jack rabbits, hares, cottontails, snowshoe rabbits, and pikas. Chickens (Gallus gallus) are an example of an avian species with which the invention may be performed.

Cells useful for the methods of the invention may be stored before use. Methods and protocols for preserving and storing of eukaryotic cells, and in particular mammalian cells, are well known in the art (cf., for example, Pollard, J. W. and Walker, J. M. (1997) Basic Cell Culture Protocols, Second Edition, Humana Press, Totowa, N.J.; Freshney, R. I. (2000) Culture of Animal Cells, Fourth Edition, Wiley-Liss, Hoboken, N.J.). Any method maintaining the biological activity of the isolated stem cells such as mesenchymal stem/progenitor cells, or progeny thereof, may be utilized in connection with the present invention. In one preferred embodiment, the cells are maintained and stored by using cryo-preservation.

In an embodiment, the cells are allogeneic or autologous.

Examples of other cell types that can be used to treat or prevent eye diseases include, but are not limited to, the cells described in WO 07/130,060 (adult retinal stem cells from extra-retinal tissues), US 2008089868 (retinal stem cells), US 2001031256 (neural retinal cells and porcine retinal pigment epithelium cells), US2006002900 (retinal pigment epithelial cells), US 2007248644 (Muller stem cells) and U.S. Pat. No. 6,162,428 (hNT-Neuron cells).

Examples of other cells types which can be used for the methods of the invention include, but are not limited to, CD34+ hemopoeitic stem cells, adipose tissue derived cells, STRO-1⁻ bone marrow derived MPCs, embryonic stem cells, and bone marrow or peripheral blood mononuclear cells.

### Cell-Sorting Techniques

Cells useful for the methods of the invention can be obtained using a variety of techniques. For example, a number of cell-sorting techniques by which cells are physically separated by reference to a property associated with the cell-antibody complex, or a label attached to the antibody can be used. This label may be a magnetic particle or a fluorescent molecule. The antibodies may be cross-linked such that they form aggregates of multiple cells, which are separable by their density. Alternatively the antibodies may be attached to a stationary matrix, to which the desired cells adhere.

In a preferred embodiment, an antibody (or other binding agent) that binds TNAP⁺, STRO-1⁺, VCAM-1⁺, THY-1⁺, STRO-2⁺, 3G5⁺, CD45⁺, CD146⁺ is used to isolate the cells. More preferably, an antibody (or other binding agent) that binds TNAP⁺ or STRO-1⁺ is used to isolate the cells.

Various methods of separating antibody-bound cells from unbound cells are known. For example, the antibody bound to the cell (or an anti-isotype antibody) can be labelled and then the cells separated by a mechanical cell sorter that detects the presence of the label. Fluorescence-activated cell sorters are well known in the art. In one embodiment, anti-TNAP antibodies and/or an STRO-1 antibodies are attached to a solid support. Various solid supports are known to those of skill in the art, including, but not limited to, agarose beads, polystyrene beads, hollow fiber membranes, polymers, and plastic petri dishes. Cells that are bound by the antibody can be removed from the cell suspension by simply physically separating the solid support from the cell suspension.

Super paramagnetic microparticles may be used for cell separations. For example, the microparticles may be coated with anti-TNAP antibodies and/or STRO-1 antibodies. The antibody-tagged, super paramagnetic microparticles may then be incubated with a solution containing the cells of interest. The microparticles bind to the surfaces of the desired stem cells, and these cells can then be collected in a magnetic field.

In another example, the cell sample is allowed to physically contact, for example, a solid phase-linked anti-TNAP monoclonal antibodies and/or anti-STRO-1 monoclonal antibodies. The solid-phase linking can comprise, for instance, adsorbing the antibodies to a plastic, nitrocellulose, or other surface. The antibodies can also be adsorbed on to the walls of the large pores (sufficiently large to permit flow-through of cells) of a hollow fiber membrane. Alternatively, the antibodies can be covalently linked to a surface or bead, such as Pharmacia Sepharose 6 MB macrobeads. The exact conditions and duration of incubation for the solid phase-linked antibodies with the stem cell containing suspension will depend upon several factors specific to the system employed. The selection of appropriate conditions, however, is well within the skill of the art.

The unbound cells are then eluted or washed away with physiologic buffer after allowing sufficient time for the stem cells to be bound. The unbound cells can be recovered and used for other purposes or discarded after appropriate testing has been done to ensure that the desired separation had been achieved. The bound cells are then separated from the solid phase by any appropriate method, depending mainly upon the nature of the solid phase and the antibody. For example, bound cells can be eluted from a plastic petri dish by vigorous agitation. Alternatively, bound cells can be eluted by enzymatically “nicking” or digesting an enzyme-sensitive “spacer” sequence between the solid phase and the antibody. Spacers bound to agarose beads are commercially available from, for example, Pharmacia.

The eluted, enriched fraction of cells may then be washed with a buffer by centrifugation and said enriched fraction may be cryopreserved in a viable state for later use according to conventional technology, culture expanded and/or introduced into the patient.

Compounds that Disrupt VEGF-Signalling

Compounds for use in the methods of the invention can be any type of molecule that decreases the ability of a VEGF to exert its normal biological effect. For example, the compound may bind, or reduce the production of, the VEGF per se, a receptor thereof, or an intracellular signalling protein or transcription factor activated and/or synthesized upon VEGF receptor activation following binding by a VEGF. Thus, as used herein, the term “compound that disrupts VEGF-signalling” refers to the compound that reduces the amount of a VEGF, a VEGF receptor or other molecule involved in VEGF-signalling, and/or the ability of a VEGF to signal through its corresponding receptor and produce the relevant downstream biological effect such as promoting cell growth and/or division.

The binding between a compound and its target (for example, VEGF) may be mediated by covalent or non-covalent interactions or a combination of covalent and non-covalent interactions. When the interaction produces a non-covalently bound complex, the binding which occurs is typically electrostatic, hydrogen-bonding, or the result of hydrophilic/lipophilic interactions. In one embodiment, the compound is a purified and/or recombinant polypeptide. Particularly preferred compounds are purified and/or recombinant antibodies, antibody-related molecules or antigenic binding fragments thereof.

Although not essential, the compound may bind specifically to the target. The phrase “specifically binds”, means that under particular conditions, the compound binds the target and does not bind to a significant amount to other, for example, proteins or carbohydrates. For example, in an embodiment the compound specifically binds VEGF-A, but does not bind other VEGFs. In another embodiment, a compound is considered to “specifically bind” if there is a greater than 10 fold difference, and preferably a 25, 50 or 100 fold greater difference between the binding of the compound to the target when compared to another protein.

Examples of compounds useful for the invention include, but are not limited to, quinazoline derivative inhibitors of VEGFs (US 2007265286, US 2003199491 and U.S. Pat. No. 6,809,097), quercetin (inhibits VEGFs) (WO 02/057473), quinazoline derivative inhibitors of VEGFR tyrosine kinases (US 2007027145), aminobenzoic acid derivative inhibitors of VEGFR tyrosine kinases (U.S. Pat. No. 6,720,424), pyridine derivative inhibitors of VEGFR tyrosine kinases (US 2003158409), Recentin (Astra Zeneca) (inhibits all three VEGFRs) (WO 07/060,402), Sunitinib (Novartis) (inhibits all three VEGFRs) (WO 08/031,835 and U.S. Pat. No. 6,573,293), Pegaptanib (Macugen™) (U.S. Pat. No. 6,051,698), Axitinib (Pfizer) (inhibits all three VEGFRs) (WO 2004/087152), Sorafenib (Bayer Pharmaceuticals) (WO 07/053,573), VEGFR-1 binding peptides (US 2005100963), arginine-rich anti-vascular endothelial growth factor peptides that block VEGF binding to receptors (U.S. Pat. No. 7,291,601), VEGF-Trap (Regeneron Pharmaceuticals) (US 2005032699), soluble VEGF receptors (US2006110364 and Tseng et al., 2002), VEGF-C and VEGF-D peptidomimetic inhibitors (US 2002065218), PAI-1 which blocks release of VEGF from VEGF-heparin complex (US 2004121955), inhibitors described in US 2002068697, WO 02/081520, US 20060234941, US 2002058619, as well as further examples outlined below. In a preferred embodiment, the compound is Lucentis, Avastin or VEGF-Trap.

### Examples of Target Molecules

In an embodiment, the target molecule of the compound for disrupting VEGF-signalling is a vascular endothelial growth factor.

As used herein, the term “vascular endothelial growth factor” or “VEGF” refers to a family of growth factors which bind to tyrosine kinase receptors (VEGF receptors, or VEGFRs) on the cell surface to stimulate angiogenesis, vasculogenesis and endothelial cell growth (see, for example, Breen, 2007).

As used herein, the term “VEGF-A” refers to a member of the VEGF polypeptide growth factor family which binds to VEGFR-1 and VEGFR-2 receptors to stimulate endothelial cell mitogenesis and cell migration, stimulates MMOP activity, increases αvβ3 activity, promotes the creation and fenestration of blood vessel lumen, is chemotactic for macrophages and granulocytes, and is also a potent vasodilator (Breen, 2007; Eremina and Quaggin, 2004). Alternatively spliced transcript variants of VEGF-A have been identified which give rise to multiple different isoforms of VEGF-A. An example of a VEGF-A polypeptide includes proteins comprising an amino acid sequence provided in SEQ ID NO:1, as well as variants and/or mutants thereof. Furthermore, an example of an open reading frame encoding a preproVEGF-A is provided as SEQ ID NO:9.

As used herein, the term “VEGF-B” refers to a member of the VEGF polypeptide growth factor family which binds to VEGFR-1 receptor to stimulate angiogenesis, endothelial cell mitogenesis and migration (Breen, 2007; Olofsson et al., 1996). Alternatively spliced transcript variants of VEGF-B have been identified which give rise to several isoforms of VEGF-B. An example of a VEGF-B polypeptide includes proteins comprising an amino acid sequence provided in SEQ ID NO:2, as well as variants and/or mutants thereof. Furthermore, an example of an open reading frame encoding a preproVEGF-B is provided as SEQ ID NO:10.

As used herein, the term “VEGF-C” refers to a member of the VEGF polypeptide growth factor family which binds to VEGFR-2 and Flt4 receptors to stimulate endothelial cell mitogenesis and migration, and lymphangiogenesis (Breen, 2007; Su et al., 2007). VEGF-C undergoes a complex proteolytic maturation to generate several isoforms and only the fully processed forms can bind and activate its cognate VEGFR-2 receptors. An example of a VEGF-C polypeptide includes proteins comprising an amino acid sequence provided in SEQ ID NO:3, as well as variants and/or mutants thereof. Furthermore, an example of an open reading frame encoding a preproVEGF-C is provided as SEQ ID NO:11.

As used herein, the term “VEGF-D” refers to a member of the VEGF polypeptide growth factor family which binds to VEGFR-2 and VEGFR-3 receptors to stimulate angiogenesis, lymphangiogenesis, and endothelial cell mitogenesis and migration. VEGF-D undergoes a complex proteolytic maturation to generate several isoforms and only the fully processed forms can bind and activate its cognate VEGFR-2 and VEGFR-3 receptors. An example of a VEGF-D polypeptide includes proteins comprising an amino acid sequence provided in SEQ ID NO:4, as well as variants and/or mutants thereof. Furthermore, an example of an open reading frame encoding a preproVEGF-D is provided as SEQ ID NO:12.

In an embodiment, the target molecule for disrupting VEGF-signalling is a vascular endothelial growth factor receptor.

As used herein, the term “VEGFR-1” (also known as Flt-1) refers to member 1 of the VEGF tyrosine kinase receptor family located on the cell surface, which contains seven extracellular immunoglobulin-like domains, a single transmembrane domain and an intracellular domain containing a tyrosine kinase function, to which VEGF-A and VEGF-B bind (Olsson et al., 2006; Cross et al., 2003). Upon binding of ligand (for example VEGF-A), the VEGFR-1 receptor dimerizes and becomes activated through transphosphorylation to stimulate angiogenesis, vasculogenesis and endothelial cell growth. An example of a VEGFR-1 polypeptide includes proteins comprising an amino acid sequence provided in SEQ ID NO:5, as well as variants and/or mutants thereof. Furthermore, an example of an open reading frame encoding a VEGFR-1 is provided as SEQ ID NO:13.

As used herein, the term “VEGFR-2” (also known as KDR or Flk-1) refers to member 2 of the VEGF tyrosine kinase receptor family located on the cell surface, which contains seven extracellular immunoglobulin-like domains, a single transmembrane domain and an intracellular domain containing a tyrosine kinase function, to which VEGF-A, VEGF-C and VEGF-D bind (Olsson et al., 2006; Cross et al., 2003). Upon binding of ligand, the VEGFR-2 receptor dimerizes and becomes activated through transphosphorylation to stimulate angiogenesis, vasculogenesis and endothelial cell growth. An example of a VEGFR-2 polypeptide includes proteins comprising an amino acid sequence provided in SEQ ID NO:6, as well as variants and/or mutants thereof. Furthermore, an example of an open reading frame encoding a VEGFR-2 is provided as SEQ ID NO:14.

As used herein, the term “VEGFR-3” (also known as Flt-4) refers to member 3 of the VEGF tyrosine kinase receptor family located on the cell surface, which contains seven extracellular immunoglobulin-like domains, a single transmembrane domain and an intracellular domain containing a tyrosine kinase function, to which VEGF-C and VEGF-D bind (Olsson et al., 2006; Cross et al., 2003). Upon binding of ligand, the VEGFR-3 receptor dimerizes and becomes activated through transphosphorylation to mediate lymphangiogenesis. An example of a VEGFR-3 polypeptide includes proteins comprising an amino acid sequence provided in SEQ ID NO:7, as well as variants and/or mutants thereof. Furthermore, an example of an open reading frame encoding a VEGFR-3 is provided as SEQ ID NO:15.

In a further embodiment, the target molecule for disrupting VEGF-signalling reduces the production of a vascular endothelial growth factor. For example, the target can be hypoxia-inducible factor 1 (HIF-1).

As used herein, the term “hypoxia-inducible factor 1” (1-HIF-1) refers to a transcription factor that regulates genes involved in the response to hypoxia. For example, HIF-1 is known to upregulate VEGF expression in response to hypoxia (Zhang et al., 2007). HIF-1α is the inducible subunit of HIF-1. An example of a HIF-1 polypeptide includes proteins comprising an amino acid sequence provided in SEQ ID NO:8, as well as variants and/or mutants thereof. Furthermore, an example of an open reading frame encoding HIF-1 is provided as SEQ ID NO:16.

Examples of compounds which target HIF-1 include, but are not limited to, echinomycin (Kong et al., 2005), BDDF-1 (WO 08/004,798), S-2-amino-3-[4′-N,N,-bis(2-chloroethyl)amino]phenyl propionic acid N-oxide dihydrochloride (PX-478) (US 2005049309), chetomin (Kung et al., 2004), 3-(5′-hydroxymethyl-2′-furyl)-1-benzylindazole (YC-1) (Yeo et al., 2003), 103D5R (Tan et al., 2005), quinocarmycin monocitrate and derivatives thereof (Rapisarda et al., 2002), 3-(5′-hydroxymethyl-2′-furyl)-1-benzylindazole (US 2004198798), and NSC-134754 and NSC-643735 (Chau et al., 2005).

In a further embodiment, the target molecule for disrupting VEGF-signalling is an intracellular signalling protein or transcription factor activated and/or synthesized upon VEGF receptor activation following binding by a VEGF.

### Antibodies—General

Antibodies may exist as intact immunoglobulins, or as modifications in a variety of forms including, for example, but not limited to, domain antibodies including either the V_{H} or V_{L} domain, a dimer of the heavy chain variable region (VHH, as described for a camelid), a dimer of the light chain variable region (VLL), Fv fragments containing only the light and heavy chain variable regions, or Fd fragments containing the heavy chain variable region and the CH1 domain. A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Non-naturally occurring forms of antibodies which comprise at least one CDR, more preferably at least one variable domain, are also referred to herein as “antibody-related molecules”. Also encompassed are fragments of antibodies such as Fab, (Fab′)₂ and FabFc₂ fragments which contain the variable regions and parts of the constant regions. CDR-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric (Morrison et al., 1984) or humanized (Jones et al., 1986). As used herein the term “antibody” includes these various forms. Using the guidelines provided herein and those methods well known to those skilled in the art which are described in the references cited above and in such publications as Harlow & Lane (supra) the antibodies for use in the methods of the present invention can be readily made.

The antibodies may be Fv regions comprising a variable light (V_{L}) and a variable heavy (V_{H}) chain. The light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

A variety of immunoassay formats may be used to select antibodies specifically immunoreactive with a target molecule such as a VEGF or a receptor thereof. For example, surface labelling and flow cytometric analysis or solid-phase ELISA immunoassays are routinely used to select antibodies specifically immunoreactive with a protein or carbohydrate. See Harlow & Lane (supra) for a description of immunoassay formats and conditions that can be used to determine specific immunoreactivity.

Examples of antibodies, antibody-related molecules or fragments thereof which can be used in the methods of the invention include, but are not limited to, anti-VEGF-A antibodies such as bevacizumab (Avastin) (U.S. Pat. No. 6,054,297), ranibizumab (Lucentis) (U.S. Pat. No. 6,407,213) and those described in U.S. Pat. No. 5,730,977 and US 2002032315; anti-VEGF-B antibodies such as those described in US 2004005671 and WO 07/140,534; anti-VEGF-C antibodies such as those described in U.S. Pat. No. 6,403,088; anti-VEGF-D antibodies such as those described in U.S. Pat. No. 7,097,986; anti-VEGFR-1 antibodies such as those described in US 2003088075; anti-VEGFR-2 antibodies such as those described in U.S. Pat. No. 6,344,339, WO 99/40118 and US 2003176674); and anti-VEGFR-3 antibodies such as those described in U.S. Pat. No. 6,824,777.

### Monoclonal Antibodies

The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against target epitopes can be screened for various properties; i.e. for isotype and epitope affinity.

Animal-derived monoclonal antibodies can be used for both direct in vivo and extracorporeal immunotherapy. However, it has been observed that when, for example, mouse-derived monoclonal antibodies are used in humans as therapeutic agents, the patient produces human anti-mouse antibodies. Thus, animal-derived monoclonal antibodies are not preferred for therapy, especially for long term use. With established genetic engineering techniques it is possible, however, to create chimeric or humanized antibodies that have animal-derived and human-derived portions. The animal can be, for example, a mouse or other rodent such as a rat.

If the variable region of the chimeric antibody is, for example, mouse-derived while the constant region is human-derived, the chimeric antibody will generally be less immunogenic than a “pure” mouse-derived monoclonal antibody. These chimeric antibodies would likely be more suited for therapeutic use, should it turn out that “pure” mouse-derived antibodies are unsuitable.

Methodologies for generating chimeric antibodies are available to those in the art. For example, the light and heavy chains can be expressed separately, using, for example, immunoglobulin light chain and immunoglobulin heavy chains in separate plasmids. These can then be purified and assembled in vitro into complete antibodies; methodologies for accomplishing such assembly have been described (see, for example, Sun et al., 1986). Such a DNA construct may comprise DNA encoding functionally rearranged genes for the variable region of a light or heavy chain of an antibody linked to DNA encoding a human constant region. Lymphoid cells such as myelomas or hybridomas transfected with the DNA constructs for light and heavy chain can express and assemble the antibody chains.

In vitro reaction parameters for the formation of IgG antibodies from reduced isolated light and heavy chains have also been described. Co-expression of light and heavy chains in the same cells to achieve intracellular association and linkage of heavy and light chains into complete H2L2 IgG antibodies is also possible. Such co-expression can be accomplished using either the same or different plasmids in the same host cell.

In another preferred embodiment of the present invention the antibody is humanized, that is, an antibody produced by molecular modeling techniques wherein the human content of the antibody is maximised while causing little or no loss of binding affinity attributable to the variable region of, for example, a parental rat, rabbit or murine antibody. The methods described below are applicable to the humanisation of antibodies.

There are several factors to consider in deciding which human antibody sequence to use during the humanisation. The humanisation of light and heavy chains are considered independently of one another, but the reasoning is basically similar for each.

This selection process is based on the following rationale: A given antibody's antigen specificity and affinity is primarily determined by the amino acid sequence of the variable region CDRs. Variable domain framework residues have little or no direct contribution. The primary function of the framework regions is to hold the CDRs in their proper spatial orientation to recognize antigen. Thus the substitution of animal, for example, rodent CDRs into a human variable domain framework is most likely to result in retention of their correct spatial orientation if the human variable domain framework is highly homologous to the animal variable domain from which they originated. A human variable domain should preferably be chosen therefore that is highly homologous to the animal variable domain(s). A suitable human antibody variable domain sequence can be selected as follow.

Step 1. Using a computer program, search all available protein (and DNA) databases for those human antibody variable domain sequences that are most homologous to the animal-derived antibody variable domains. The output of a suitable program is a list of sequences most homologous to the animal-derived antibody, the percent homology to each sequence, and an alignment of each sequence to the animal-derived sequence. This is done independently for both the heavy and light chain variable domain sequences. The above analyses are more easily accomplished if only human immunoglobulin sequences are included.

Step 2. List the human antibody variable domain sequences and compare for homology. Primarily the comparison is performed on length of CDRs, except CDR3 of the heavy chain which is quite variable. Human heavy chains and Kappa and Lambda light chains are divided into subgroups; Heavy chain 3 subgroups, Kappa chain 4 subgroups, Lambda chain 6 subgroups. The CDR sizes within each subgroup are similar but vary between subgroups. It is usually possible to match an animal-derived antibody CDR to one of the human subgroups as a first approximation of homology. Antibodies bearing CDRs of similar length are then compared for amino acid sequence homology, especially within the CDRs, but also in the surrounding framework regions. The human variable domain which is most homologous is chosen as the framework for humanisation.

### The Actual Humanising Methodologies/Techniques

An antibody may be humanized by grafting the desired CDRs onto a human framework according to EP-A-0239400. A DNA sequence encoding the desired reshaped antibody can therefore be made beginning with the human DNA whose CDRs it is wished to reshape. The animal-derived variable domain amino acid sequence containing the desired CDRs is compared to that of the chosen human antibody variable domain sequence. The residues in the human variable domain are marked that need to be changed to the corresponding residue in the animal to make the human variable region incorporate the animal-derived CDRs. There may also be residues that need substituting in, adding to or deleting from the human sequence.

Oligonucleotides are synthesized that can be used to mutagenize the human variable domain framework to contain the desired residues. Those oligonucleotides can be of any convenient size. One is normally only limited in length by the capabilities of the particular synthesizer one has available. The method of oligonucleotide-directed in vitro mutagenesis is well known.

Alternatively, humanisation may be achieved using the recombinant polymerase chain reaction (PCR) methodology of WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

Following the mutagenesis reactions to reshape the antibody, the mutagenised DNAs can be linked to an appropriate DNA encoding a light or heavy chain constant region, cloned into an expression vector, and transfected into host cells, preferably mammalian cells. These steps can be carried out in routine fashion. A reshaped antibody may therefore be prepared by a process comprising:
(a) preparing a first replicable expression vector including a suitable promoter operably linked to a DNA sequence which encodes at least a variable domain of an Ig heavy or light chain, the variable domain comprising framework regions from a human antibody and the CDRs required for the humanized antibody of the invention;
(b) preparing a second replicable expression vector including a suitable promoter operably linked to a DNA sequence which encodes at least the variable domain of a complementary Ig light or heavy chain respectively;
(c) transforming a cell line with the first or both prepared vectors; and
(d) culturing said transformed cell line to produce said altered antibody.

Preferably the DNA sequence in step (a) encodes both the variable domain and each constant domain of the human antibody chain. The humanized antibody can be prepared using any suitable recombinant expression system. The cell line which is transformed to produce the altered antibody may be a Chinese Hamster Ovary (CHO) cell line or an immortalised mammalian cell line, which is advantageously of lymphoid origin, such as a myeloma, hybridoma, trioma or quadroma cell line. The cell line may also comprise a normal lymphoid cell, such as a B-cell, which has been immortalised by transformation with a virus, such as the Epstein-Barr virus. Most preferably, the immortalised cell line is a myeloma cell line or a derivative thereof.

The CHO cells used for expression of the antibodies may be dihydrofolate reductase (dhfr) deficient and so dependent on thymidine and hypoxanthine for growth. The parental dhfr CHO cell line is transfected with the DNA encoding the antibody and dhfr gene which enables selection of CHO cell transformants of dhfr positive phenotype. Selection is carried out by culturing the colonies on media devoid of thymidine and hypoxanthine, the absence of which prevents untransformed cells from growing and transformed cells from resalvaging the folate pathway and thus bypassing the selection system. These transformants usually express low levels of the DNA of interest by virtue of co-integration of transfected DNA of interest and DNA encoding dhfr. The expression levels of the DNA encoding the antibody may be increased by amplification using methotrexate (MTX). This drug is a direct inhibitor of the enzyme dhfr and allows isolation of resistant colonies which amplify their dhfr gene copy number sufficiently to survive under these conditions. Since the DNA sequences encoding dhfr and the antibody are closely linked in the original transformants, there is usually concomitant amplification, and therefore increased expression of the desired antibody.

Another preferred expression system for use with CHO or myeloma cells is the glutamine synthetase (GS) amplification system described in WO 87/04462. This system involves the transfection of a cell with DNA encoding the enzyme GS and with DNA encoding the desired antibody. Cells are then selected which grow in glutamine free medium and can thus be assumed to have integrated the DNA encoding GS. These selected clones are then subjected to inhibition of the enzyme GS using methionine sulphoximine (Msx). The cells, in order to survive, will amplify the DNA encoding GS with concomitant amplification of the DNA encoding the antibody.

Although the cell line used to produce the humanized antibody is preferably a mammalian cell line, any other suitable cell line, such as a bacterial cell line or a yeast cell line, may alternatively be used. In particular, it is envisaged that E. coli-derived bacterial strains could be used. The antibody obtained is checked for functionality. If functionality is lost, it is necessary to return to step (2) and alter the framework of the antibody.

Once expressed, the whole antibodies, their dimers, individual light and heavy chains, or other immunoglobulin forms can be recovered and purified according to standard procedures of the art, including ammonium sulfate precipitation, affinity columns, column chromatography, gel electrophoresis and the like (See, generally, Scopes, R., Protein Purification, Springer-Verlag, N.Y. (1982)). Substantially pure immunoglobulins of at least about 90 to 95% homogeneity are preferred, and 98 to 99% or more homogeneity most preferred, for pharmaceutical uses. Once purified, partially or to homogeneity as desired, a humanized antibody may then be used therapeutically or in developing and performing assay procedures, immunofluorescent stainings, and the like (See, generally, Lefkovits and Pernis (editors), Immunological Methods, Vols. I and II, Academic Press, (1979 and 1981)).

Antibodies with fully human variable regions can also be prepared by administering the antigen to a transgenic animal which has been modified to produce such antibodies in response to antigenic challenge, but whose endogenous loci have been disabled. Various subsequent manipulations can be performed to obtain either antibodies per se or analogs thereof (see, for example, U.S. Pat. No. 6,075,181).

### Gene Silencing

In an embodiment, VEGF-signalling is disrupted using gene silencing. The terms “RNA interference”, “RNAi” or “gene silencing” refers generally to a process in which a double-stranded RNA (dsRNA) molecule reduces the expression of a nucleic acid sequence with which the double-stranded RNA molecule shares substantial or total homology. However, it has more recently been shown that gene silencing can be achieved using non-RNA double stranded molecules (see, for example, US 20070004667).

RNA interference (RNAi) is particularly useful for specifically inhibiting the production of a particular RNA and/or protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029 and WO 01/34815.

The present invention includes the use of nucleic acid molecules comprising and/or encoding double-stranded regions for gene silencing. The nucleic acid molecules are typically RNA but may comprise DNA, chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The % identity of a nucleic acid molecule is determined by GAP (Needleman and Wunsch, 1970) analysis (GCG program) with a gap creation penalty=5, and a gap extension penalty=0.3. Preferably, the two sequences are aligned over their entire length.

The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNAi), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression.

Preferred small interfering RNA (‘siRNA“) molecules comprise a nucleotide sequence that is identical to about 19 to 23 contiguous nucleotides of the target mRNA. In an embodiment, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the avain (preferably chickens) in which it is to be introduced, e.g., as determined by standard BLAST search.

By “shRNA” or “short-hairpin RNA” is meant an siRNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to 15 nucleotides which forms a single-stranded loop above the stem structure created by the two regions of base complementarity. Examples of sequences of a single-stranded loops are 5′ UUCAAGAGA 3′ and 5′ UUUGUGUAG 3′.

Included shRNAs are dual or bi-finger and multi-finger hairpin dsRNAs, in which the RNA molecule comprises two or more of such stem-loop structures separated by single-stranded spacer regions.

There are well-established criteria for designing siRNAs (see, for example, Elbashire et al., 2001; Amarzguioui et al., 2004; Reynolds et al., 2004). Details can be found in the websites of several commercial vendors such as Ambion, Dharmacon, GenScript, and OligoEngine. Typically, a number of siRNAs have to be generated and screened in order to compare their effectiveness.

Once designed, the dsRNAs for use in the method of the present invention can be generated by any method known in the art, for example, by in vitro transcription, recombinantly, or by synthetic means. siRNAs can be generated in vitro by using a recombinant enzyme, such as T7 RNA polymerase, and DNA oligonucleotide templates, or can be prepared in vivo, for example, in cultured cells. In a preferred embodiment, the nucleic acid molecule is produced synthetically.

In addition, strategies have been described for producing a hairpin siRNA from vectors containing, for example, a RNA polymerase III promoter. Various vectors have been constructed for generating hairpin siRNAs in host cells using either an H1-RNA or an snU6 RNA promoter. A RNA molecule as described above (e.g., a first portion, a linking sequence, and a second portion) can be operably linked to such a promoter. When transcribed by RNA polymerase III, the first and second portions form a duplexed stem of a hairpin and the linking sequence forms a loop. The pSuper vector (OligoEngines Ltd., Seattle, Wash.) also can be used to generate siRNA.

Modifications or analogs of nucleotides can be introduced to improve the properties of the nucleic acid molecules of the invention. Improved properties include increased nuclease resistance and/or increased ability to permeate cell membranes. Accordingly, the terms “polynucleotide” and “double-stranded RNA molecule” etc includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In an embodiment, the ds molecule, preferably dsRNA, comprises an oligonucleotide which comprises at least 19 contiguous nucleotides of any one or more of the sequence of nucleotides provided as SEQ ID NOs 9 to 16 where T is replaced with a U, wherein the portion of the molecule that is double stranded is at least 19 basepairs in length and comprises said oligonucleotide.

Examples of ds molecules which can be used in the methods of the invention include, but are not limited to, those described in CN 1804038, CN 1834254, WO 08/045,576, US 2006025370, US 2006094032, GB 2406569, CA 2537085, WO 03/070910, US 2006217332, US 2005222066, US 2005054596, US 2004209832 and US 2004138163, US 2005148530 and US 2005171039.

### Antisense Polynucleotides

The term “antisense polynucleotide” shall be taken to mean a DNA or RNA, or combination thereof, molecule that is complementary to at least a portion of a specific mRNA molecule encoding a polypeptide of the invention and capable of interfering with a post-transcriptional event such as mRNA translation. The use of antisense methods is well known in the art (see for example, G. Hartmann and S. Endres, Manual of Antisense Methodology, Kluwer (1999)). Senior (1998) states that antisense methods are now a very well established technique for manipulating gene expression.

An antisense polynucleotide of the invention will hybridize to a target polynucleotide under physiological conditions. As used herein, the term “an antisense polynucleotide which hybridises under physiological conditions” means that the polynucleotide (which is fully or partially single stranded) is at least capable of forming a double stranded polynucleotide with mRNA encoding a protein, such as those provided in any one of SEQ ID NOs 9 to 16 under normal conditions in a cell, preferably a human cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

Examples of antisense polynucleotides which can be used in the methods of the invention include, but are not limited to, those described in US 2003186920 and WO 07/013,704.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988; Perriman et al., 1992) and the hairpin ribozyme (Shippy et al., 1999).

The ribozymes for use in this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for a catalytic polynucleotide of the invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides of the invention should also be capable of hybridizing a target nucleic acid molecule (for example an mRNA encoding any polypeptide provided in SEQ ID NOs 1 to 8) under “physiological conditions”, namely those conditions within a cell (especially conditions in an animal cell such as a human cell).

Examples of ribozymes which can be used in the methods of the invention include, but are not limited to, those described in U.S. Pat. No. 6,346,398, Ciafre et al. (2004) and Weng et al. (2005).

### Gene Therapy

Therapeutic polynucleotides molecules described herein may be employed in accordance with the present invention by expression of such polynucleotides in treatment modalities often referred to as “gene therapy”. Thus, cells from a patient may be engineered with a polynucleotide, such as a DNA or RNA, to encode a polynucleotide ex vivo. The engineered cells can then be provided to a patient to be treated with the polynucleotide, or where relevant the polypeptide (such as an anti-VEGF antibody) encoded thereby. In this embodiment, cells may be engineered ex vivo, for example, by the use of a retroviral plasmid vector to transform, for example, stem cells or differentiated stem cells. Such methods are well-known in the art and their use in the present invention will be apparent from the teachings herein.

Further, cells may be engineered in vivo for expression of a polynucleotide in vivo by procedures known in the art. For example, a polynucleotide may be engineered for expression in a replication defective retroviral vector or adenoviral vector or other vector (e.g., poxvirus vectors). The expression construct may then be isolated. A packaging cell is transduced with a plasmid vector containing RNA encoding a polynucleotide as described herein, such that the packaging cell now produces infectious viral particles containing the gene of interest. These producer cells may be administered to a patient for engineering cells in vivo and expression of the polynucleotide in vivo. These and other methods for administering a polynucleotide should be apparent to those skilled in the art from the teachings of the present invention.

Retroviruses from which the retroviral plasmid vectors hereinabove-mentioned may be derived include, but are not limited to, Moloney Murine Leukemia Virus, Spleen Necrosis Virus, Rous Sarcoma Virus, Harvey Sarcoma Virus, Avian Leukosis Virus, Gibbon Ape Leukemia Virus, Human Immunodeficiency Virus, Adenovirus, Myeloproliferative Sarcoma Virus, and Mammary Tumor Virus. In a preferred embodiment, the retroviral plasmid vector is derived from Moloney Murine Leukemia Virus.

Such vectors will include one or more promoters for expressing the polynucleotide. Suitable promoters which may be employed include, but are not limited to, the retroviral LTR; the SV40 promoter; and the human cytomegalovirus (CMV) promoter. Cellular promoters such as eukaryotic cellular promoters including, but not limited to, the histone, RNA polymerase III, the metallothionein promoter, heat shock promoters, the albumin promoter, human globin promoters and α-actin promoters, can also be used. Additional viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The retroviral plasmid vector can be employed to transduce packaging cell lines to form producer cell lines. Examples of packaging cells which may be transfected include, but are not limited to, the PE501, PA317, Y-2, Y-AM, PA12, T19-14X, VT-19-17-H2, YCRE, YCRIP, GP+E-86, GP+envAm12, and DAN cell lines as described by Miller (1990). The vector may be transduced into the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line will generate infectious retroviral vector particles, which include the polynucleotide. Such retroviral vector particles may then be employed to transduce eukaryotic cells, either in vitro or in vivo. The transduced eukaryotic cells will express the polynucleotide, and where relevant produce the polypeptide encoded thereby. Eukaryotic cells which may be transduced include, but are not limited to, embryonic stem cells, retinal stem cells, embryonic carcinoma cells, as well as hematopoietic stem cells, hepatocytes, fibroblasts, myoblasts, keratinocytes, myocytes (particularly skeletal muscle cells), endothelial cells, and bronchial epithelial cells.

In an embodiment, the cells administered as part of the combination therapy are not genetically modified cells such that they produce the compound. In a particularly preferred embodiment, the cells administered as part of the combination therapy are not genetically modified cells such that they produce an anti-VEGF monoclonal antibody.

A selective marker may be included in the construct or vector for the purposes of monitoring successful genetic modification and for selection of cells into which a polynucleotide has been integrated. Non-limiting examples include drug resistance markers, such as G148 or hygromycin. Additionally negative selection may be used, for example wherein the marker is the HSV-tk gene. This gene will make the cells sensitive to agents such as acyclovir and gancyclovir. The NeoR (neomycin/G148 resistance) gene is commonly used but any convenient marker gene may be used whose gene sequences are not already present in the target cell can be used. Further non-limiting examples include low-affinity Nerve Growth Factor (NGFR), enhanced fluorescent green protein (EFGP), dihydrofolate reductase gene (DHFR) the bacterial hisD gene, murine CD24 (HSA), murine CD8a(lyt), bacterial genes which confer resistance to puromycin or phleomycin, and β-galactosidase.

The additional polynucleotide sequence(s) may be introduced into the cell on the same vector or may be introduced into the host cells on a second vector. In a preferred embodiment, a selective marker will be included on the same vector as the polynucleotide.

The present invention also encompasses genetically modifying the promoter region of an endogenous gene such that expression of the endogenous gene is up-regulated resulting in the increased production of the encoded protein compared to a wild type cell.

In a useful embodiment of the invention, the cells are genetically modified to contain a gene that disrupts or inhibits angiogenesis. The gene may encode a cytotoxic agent such as ricin. In another embodiment, the gene encodes a cell surface molecule that elicits an immune rejection response. For example, the cells can be genetically modified to produce α1, 3 galactosyl transferase. This enzyme synthesizes α1, 3 galactosyl epitopes that are the major xenoantigens, and its expression causes hyperacute immune rejection of the transgenic endothelial cells by preformed circulating antibodies and/or by T cell mediated immune rejection.

Genetic therapies in accordance with the present invention may involve a transient (temporary) presence of the gene therapy polynucleotide in the patient or the permanent introduction of a polynucleotide into the patient.

### Compositions and Administration Thereof

Typically, the cells and the compound are administered in a pharmaceutical composition comprising at least one pharmaceutically-acceptable carrier. Furthermore, an aspect of the invention relates to a composition comprising cells and a compound that disrupts VEGF-signalling, and optionally a pharmaceutically-acceptable carrier.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, or solvent encapsulating material.

Pharmaceutically acceptable carriers include saline, aqueous buffer solutions, solvents and/or dispersion media. The use of such carriers are well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Some examples of materials and solutions which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The pharmaceutical compositions comprising cells useful for the methods of the invention may comprise a polymeric carrier or extracellular matrix.

A variety of biological or synthetic solid matrix materials (i.e., solid support matrices, biological adhesives or dressings, and biological/medical scaffolds) are suitable for use in this invention. The matrix material is preferably medically acceptable for use in in vivo applications. Non-limiting examples of such medically acceptable and/or biologically or physiologically acceptable or compatible materials include, but are not limited to, solid matrix materials that are absorbable and/or non-absorbable, such as small intestine submucosa (SIS), e.g., porcine-derived (and other SIS sources); crosslinked or non-crosslinked alginate, hydrocolloid, foams, collagen gel, collagen sponge, polyglycolic acid (PGA) mesh, polyglactin (PGL) mesh, fleeces, foam dressing, bioadhesives (e.g., fibrin glue and fibrin gel) and dead de-epidermized skin equivalents in one or more layers.

Fibrin glues are a class of surgical sealants which have been used in various clinical settings. As the skilled address would be aware, numerous sealants are useful in compositions for use in the methods of the invention. However, a preferred embodiment of the invention relates to the use of fibrin glues with the cells described herein.

When used herein the term “fibrin glue” refers to the insoluble matrix formed by the cross-linking of fibrin polymers in the presence of calcium ions. The fibrin glue may be formed from fibrinogen, or a derivative or metabolite thereof, fibrin (soluble monomers or polymers) and/or complexes thereof derived from biological tissue or fluid which forms a fibrin matrix. Alternatively, the fibrin glue may be formed from fibrinogen, or a derivative or metabolite thereof, or fibrin, produced by recombinant DNA technology.

The fibrin glue may also be formed by the interaction of fibrinogen and a catalyst of fibrin glue formation (such as thrombin and/or Factor XIII). As will be appreciated by those skilled in the art, fibrinogen is proteolytically cleaved in the presence of a catalyst (such as thrombin) and converted to a fibrin monomer. The fibrin monomers may then form polymers which may cross-link to form a fibrin glue matrix. The cross-linking of fibrin polymers may be enhanced by the presence of a catalyst such as Factor XIII. The catalyst of fibrin glue formation may be derived from blood plasma, cryoprecipitate or other plasma fractions containing fibrinogen or thrombin. Alternatively, the catalyst may be produced by recombinant DNA technology.

The rate at which the clot forms is dependent upon the concentration of thrombin mixed with fibrinogen. Being an enzyme dependent reaction, the higher the temperature (up to 37° C.) the faster the clot formation rate. The tensile strength of the clot is dependent upon the concentration of fibrinogen used.

Use of fibrin glue and methods for its preparation and use are described in U.S. Pat. No. 5,643,192. U.S. Pat. No. 5,643,192 discloses the extraction of fibrinogen and thrombin components from a single donor, and the combination of only these components for use as a fibrin glue. U.S. Pat. No. 5,651,982, describes another preparation and method of use for fibrin glue. U.S. Pat. No. 5,651,982, provides a fibrin glue with liposomes for use as a topical sealant in mammals.

Several publications describe the use of fibrin glue for the delivery of therapeutic agents. For example, U.S. Pat. No. 4,983,393 discloses a composition for use as an intra-vaginal insert comprising agarose, agar, saline solution glycosaminoglycans, collagen, fibrin and an enzyme. Further, U.S. Pat. No. 3,089,815 discloses an injectable pharmaceutical preparation composed of fibrinogen and thrombin and U.S. Pat. No. 6,468,527 discloses a fibrin glue which facilitates the delivery of various biological and non-biological agents to specific sites within the body. Such procedures can be used in the methods of the invention.

Suitable polymeric carriers include porous meshes or sponges formed of synthetic or natural polymers, as well as polymer solutions. One form of matrix is a polymeric mesh or sponge; the other is a polymeric hydrogel. Natural polymers that can be used include proteins such as collagen, albumin, and fibrin; and polysaccharides such as alginate and polymers of hyaluronic acid. Synthetic polymers include both biodegradable and non-biodegradable polymers. Examples of biodegradable polymers include polymers of hydroxy acids such as polylactic acid (PLA), polyglycolic acid (PGA), and polylactic acid-glycolic acid (PLGA), polyorthoesters, polyanhydrides, polyphosphazenes, and combinations thereof. Non-biodegradable polymers include polyacrylates, polymethacrylates, ethylene vinyl acetate, and polyvinyl alcohols.

Polymers that can form ionic or covalently crosslinked hydrogels which are malleable are used to encapsulate cells. A hydrogel is a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are crosslinked ionically, or block copolymers such as Pluronics™ or Tetronics™, polyethylene oxide-polypropylene glycol block copolymers which are crosslinked by temperature or pH, respectively. Other materials include proteins such as fibrin, polymers such as polyvinylpyrrolidone, hyaluronic acid and collagen.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups. Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Further, a composition used for a methods of the invention may comprise at least one other therapeutic agent. For example, the composition may contain an analgesic to aid in treating inflammation or pain, another anti-angiogenic compound, or an anti-infective agent to prevent infection of the site treated with the composition. More specifically, non-limiting examples of useful therapeutic agents include the following therapeutic categories: analgesics, such as nonsteroidal anti-inflammatory drugs, opiate agonists and salicylates; anti-infective agents, such as antihelmintics, antianaerobics, antibiotics, aminoglycoside antibiotics, antifungal antibiotics, cephalosporin antibiotics, macrolide antibiotics, miscellaneous β-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, gastrointestinal anti-inflammatory agents, gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; and vitamins, such as vitamin A, vitamin B, vitamin C, vitamin D, vitamin E, and vitamin K.

Examples of other anti-angiogenic factors which may be used with the present invention, either in a single composition or as a combined therapy, include, but are not limited to, platelet factor 4; protamine sulphate; sulphated chitin derivatives (prepared from queen crab shells); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3; Chymostatin; Cyclodextrin Tetradecasulfate; Eponemycin; Camptothecin; Fumagillin; Gold Sodium Thiomalate; anticollagenase-serum; alpha2-antiplasmin; Bisantrene (National Cancer Institute); Lobenzarit disodium (N-(2)-carboxyphenyl-4-chloroanthronilic acid disodium); Thalidomide; Angostatic steroid; AGM-1470; carboxynaminolmidazole; and metalloproteinase inhibitors such as BB94.

In certain embodiments, the other therapeutic agent may be a growth factor or other molecule that affects cell differentiation and/or proliferation. Growth factors that induce final differentiation states are well-known in the art, and may be selected from any such factor that has been shown to induce a final differentiation state. Growth factors for use in methods described herein may, in certain embodiments, be variants or fragments of a naturally-occurring growth factor.

Compositions useful for the methods of the present invention comprising cells may include cell culture components, e.g., culture media including amino acids, metals, coenzyme factors, as well as small populations of other cells, e.g., some of which may arise by subsequent differentiation of the stem cells.

Compositions useful for the methods of the present invention comprising cells may be prepared, for example, by sedimenting out the subject cells from the culture medium and re-suspending them in the desired solution or material. The cells may be sedimented and/or changed out of the culture medium, for example, by centrifugation, filtration, ultrafiltration, etc.

Compositions may be administered orally, parenteral, buccal, vaginal, rectal, inhalation, insufflation, sublingually, intramuscularly, subcutaneously, topically, intranasally, intraocularly, intraperitoneally, intrathoracially, intravenously, epidurally, intrathecally, intracerebroventricularly and by injection into the joints.

Cells and/or compounds may be administered to the eye or eye lid, for example, using drops, an ointment, a cream, a gel, a suspension, an implant, etc. In another embodiment, intra-ocular injection is used to treat an eye disease. In one embodiment, cells and/or compounds may be administered intravitreally, in another embodiment, subretinally, while in another embodiment, intra-retinally, while in another embodiment, periocularly. In one embodiment, cells and/or compounds may be administered intracamerally into the anterior chamber or vitreous, via a depot attached to the intraocular lens implant inserted during surgery, or via a depot placed in the eye sutured in the anterior chamber or vitreous. The cells and/or compound may be formulated with excipients such as methylcellulose, hydroxypropyl methylcellulose, hydroxypropyl cellulose, polyvinyl pyrrolidine, neutral poly(meth)acrylate esters, and other viscosity-enhancing agents. The cells and/or compound may be injected into the eye, for example, injection under the conjunctiva or tenon capsule, intravitreal injection, or retrobulbar injection. The cells and/or compound may be administered with a slow release drug delivery system, such as polymers, matrices, microcapsules, or other delivery systems formulated from, for example, glycolic acid, lactic acid, combinations of glycolic and lactic acid, liposomes, silicone, polyanhydride polyvinyl acetate alone or in combination with polyethylene glycol, etc. The delivery device can be implanted intraocularly, for example, implanted under the conjunctiva, implanted in the wall of the eye, sutured to the sclera, for long-term drug delivery. Methods of introduction may additionally be provided by non-biodegradable devices. In particular, the cells and/or compound can be administered via an implantable lens. The cells and/or compound can be coated on the lens, dispersed throughout the lens or both.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL (BASF, Parsippany, N.J.) or phosphate buffered saline (PBS). In all cases, the composition must be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, w ater, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, sodium chloride can also be included in the composition. Prolonged absorption of the injectable compositions can be brought about by including in the composition an agent that delays absorption, such as aluminum monostearate or gelatin.

Sterile injectable solutions can be prepared by incorporating the compound and/or cells in the required amount in an appropriate solvent with one or a combination of ingredients enumerated above, as required, followed by filtered sterilization. Generally, dispersions are prepared by incorporating the polynucleotide into a sterile vehicle, which contains a basic dispersion medium and the required other ingredients from those enumerated above. In the case of sterile powders for the preparation of sterile injectable solutions, suitable methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the compound or cells can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Formulations suitable for nasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for administration by nebulizer, include aqueous or oily solutions of the agent. For administration by inhalation, the compound or cells can also be delivered in the form of drops or an aerosol spray from a pressured container or dispenser that contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, eye drops, or suppositories. For transdermal administration, the active compound is formulated into ointments, salves, gels, or creams, as generally known in the art.

The skilled artisan can readily determine the amount of cells, compounds and optional carrier(s) in compositions and to be administered in methods of the invention. In an embodiment, any additives (in addition to the active cells or compound) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

The concentration of the cells in the composition may be at least about 5×10⁵ cells/mL, at least about 1×10⁶ cells/mL, at least about 5×10⁶ cells/mL, at least about 10⁷ cells/mL, at least about 2×10⁷ cells/mL, at least about 3×10⁷ cells/mL, or at least about 5×10⁷ cells/mL.

The compound may be administered in an amount of about 0.001 to 2000 mg/kg body weight per dose, and more preferably about 0.01 to 500 mg/kg body weight per dose. Repeated doses may be administered as prescribed by the treating physician.

The present invention relates to the combined use of cells and a compound that disrupts VEGF-signalling to treat or prevent an angiogenesis-related disease. The term “in combination with” or “combined therapy” or variations thereof means the cells and compound can be administered simultaneously, either in the same composition or separately (e.g., within about 5 minutes of each other), in a sequential manner, or both, as well as temporally spaced order of up to several hours, days or weeks apart. Such combination treatment may also include more than a single administration. It is contemplated that such combination therapies may include administering one therapeutic agent multiple times between the administrations of the other. The time period between the administration may range from a few seconds (or less) to several hours or days, and will depend on, for example, the properties of cells or compounds (e.g., potency, solubility, bioavailability, half-life, and kinetic profile), as well as the condition of the patient.

In an embodiment, the compound is administered before the cells. This is particularly the case if the agent binds a VEGF or a receptor thereof. In an embodiment, the compound is administered about 1 day, 3 days, 5 days, 7 days, 9 days, or 14 days, before the cells.

The methods of the invention may be combined with other therapies for treating or preventing an eye disease and/or an angiogenesis-related disease. The nature of these other therapies will depend on the particular angiogenesis-related disease. For example, for the treatment or prevention of macular degeneration using the methods of the invention may be combined with antioxidant and/or zinc supplements, administration of macugen (Pegaptanib), using a method as defined in U.S. Pat. No. 6,942,655, steroid therapy and/or laser treatment (such as Visudyne™). With regard to cancer, treatment with the methods of the invention can be combined with surgery, radiation therapy and/or chemotherapy.

### Example

The invention is hereinafter described by way of the following non-limiting Examples and with reference to the accompanying figures.

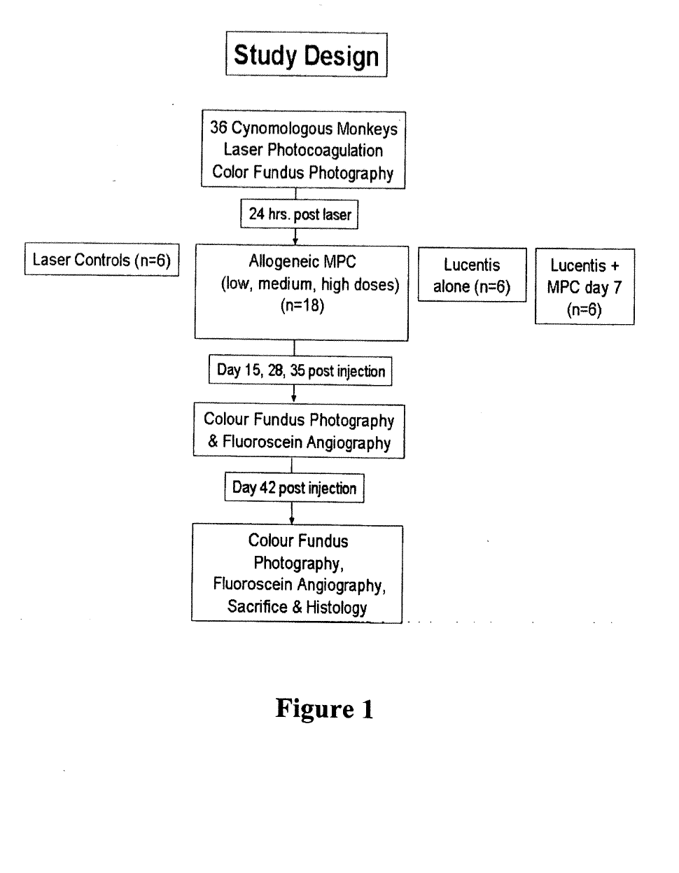

A summary of the design of the study is provided as FIG. 1.

Materials and Methods

Receipts

| | |
|---|---|
| Species | Macaca fascicularis |
| Strain | Cynomolgus |
| Source | PCS Preferred Supplier. |
| Age | Approximately 1.5 to 3.5 years old at the |
| | onset of treatment |
| Weight Range | Approximately 2 to 4 kg at the onset of treatment |
| No. of Groups | 7 |
| No. of Animals | 6 males/group and 2 spares (total 44 animals) |

### Housing

Animals were group housed (2 or 3) when possible, in stainless steel cages equipped with a bar-type floor and an automatic watering valve. Each cage was clearly labeled with a color-coded cage card indicating project, group, animal number and tattoo.

Each animal was uniquely identified by a permanent skin tattoo. The targeted conditions for animal room environment and photoperiod are as follows:

| | |
|---|---|
| Temperature | 24 ± 3° C. |
| Humidity | 50 ± 20% |
| Light cycle | 12 h light and 12 h dark |
| | (except during designated procedures). |

### Dietary Materials

All animals had access to a standard certified pelleted commercial primate food (2050C Certified Global 20% Protein Primate Diet: Harlan) twice daily except during designated procedures. In addition, each animal was offered food supplements daily in any combination of the following: Golden Banana Sofly®, Prima-Treat® (5 g format) and/or fresh or dried fruit and at least once weekly Prima-Foraging Crumbles® as part of the environmental enrichment program. Additional fruit supplements were provided following anesthetic recovery to stimulate appetite and maintain nutrition.

Maximum allowable concentrations of contaminants in the diet (e.g., heavy metals, aflatoxin, organophosphate, chlorinated hydrocarbons, PCBs) were controlled and routinely analyzed by the manufacturers. Municipal tap water which had been softened, purified by reverse osmosis and exposed to ultraviolet light was freely available (except during designated procedures). It is considered that there were no known contaminants in the dietary materials that could interfere with the objectives of the study.

### Assignment to Groups

Prior to treatment initiation, animals were assigned to the treatment groups using a computer-based randomization procedure that uses stratification with body weight as the parameter (animals in poor health were assigned to groups) (Table 1).

### Preparation of Cells

Simian Marrow Progenitor Cells—Cynomolgus Monkey (smMPC-cyno) (also referred to in this Example as MPCs) were isolated from ˜15 ml of bone marrow aspirate collected from a female Macaca fascicularis (D.O.B. Mar. 12, 2005) on Jun. 25, 2007 per Master Batch Record 3001.MES. The marrow aspirate suspension was Ficolled and washed to remove non-nucleated cells (red blood cells). The nucleated cells were counted then separated by attaching CA12 antibody (also known as the STRO-3 antibody—see WO 2006/108229) and Dynalbeads. The cells with antibody and beads attached were positively selected by the magnetic field of an MPC-1 magnet. The positive selected cells were counted and seeded into T-flasks at p.0 in Growth Medium. Pre-selection, Positive, and Negative cells were used in a colony forming assay (CFU-F).

TABLE 1
Allocated of animals.

| | Dose | | Dose | | Laser | Termination | Population |
|---|---|---|---|---|---|---|---|
| Groups | Level/eye | | Volume | Dosing | Schedule | Date | ♂ |
| 1 - Control | 0 | cells | 50 μL | Day 1 | Day 1 | Day 43 | 6 |
| Group | | | | | | | |
| 2 - Low | 78,100 | cells | 50 μL | Day 1 | Day 1 | Day 43 | 6 |
| Dose | | | | | | | |
| 3 - Mid | 312,500 | cells | 50 μL | Day 1 | Day 1 | Day 43 | 6 |
| Dose | | | | | | | |
| 4 - High | 1,250,000 | cells | 50 μL | Day 1 | Day 1 | Day 43 | 6 |
| Dose | | | | | | | |
| 5 - Lucentis | 0.5 | mg | 50 μL | Day 1 | Day 1 | Day 43 | 6 |
| alone | | | | | | | |
| 6 - Lucentis + | 1,250,000 cells + | | 50 μL + | Day 1 | Day 1 | Day 43 | 6 |
| high | 0.5 mg | | 50 μL | | | | |
| dose** | | | | | | | |
| 7 - High | 1,250,000 | cells | 50 μL | Day 1 | - | Day 43 | 6 |
| Dose* | | | | | | | |

*Group 7 did not receive any laser treatment.
**Lucentis was administered at time of laser injury 50ul, and high dose of MPCs are administered 7 days after.

The smMPC-cyno cells were fed with Growth Media. All cultures (p.0-p.5) were fed every 2 to 4 days until they reached desired confluence. The cells were then passaged or harvested using HBSS wash and then collagenase followed by Trypsin/Versene. The p.1 cells were counted and seeded into T-flasks. When the p.1 smMPC-cyno reached desired confluence the cells were harvested and cryopreserved using a controlled rate freezer.

Passage 1 cryopreserved smMPC-cyno were thawed and seeded into T-flasks (p.2). The p.2 cells were passaged into a Cell Factory at p.3. The p.3 cells were harvested and passaged to p.4 in to a Cell Factory. Extra p.3 cells were cryopreserved. The p.4 cells were passaged to 6× Cell Factories at p.5. When the p.5 smMPC-cyno reached desired confluence the cells were harvested and cryopreserved using a controlled rate freezer. The cells were cryopreserved in 50% AlphaMEM, 42.5% Profreeze, and 7.5% DMSO (Table 2 and 3). Samples were tested for CFU-F assay, FACS, sterility, mycoplasma, and endotoxin (Table 4).

TABLE 2
smMPC-cyno 15897 amps cryopreserved at p.5.

| Cells/Amp | Number of Amps | Volume/Amp (ml) |
|---|---|---|
| 0.781 × 10⁶ | 32 | 0.5 |
| 3 × 10⁶ | 31 | 0.5 |
| 12.5 × 10⁶ | 32 | 0.5 |
| 25 × 10⁶ | 32 | 0.5 |

TABLE 3
Post-freeze cell numbers.

| | | Post-Freeze | | |
|---|---|---|---|---|
| Pre-Freeze | Post-Freeze | Viable | | Seeding |
| cells/amp | Total cells/amp | cells/amp | % Viable | Efficiency |
| 0.781 × 10⁶ | 0.642 × 10⁶ | 0.630 × 10⁶ | 98.1% | 70.5% |
| 3 × 10⁶ | 2.52 × 10⁶ | 2.49 × 10⁶ | 97.6% | 61.0% |
| 12.5 × 10⁶ | 12.9 × 10⁶ | 12.7 × 10⁶ | 98.4% | 58.3% |
| 25 × 10⁶ | 27.4 × 10⁶ | 26.8 × 10⁶ | 97.8% | 52.8% |

TABLE 4
Test results.

| Test | Result |
|---|---|
| CFU-F assay | 5.84 fold CA 12+ increase |
| CA12 | 3.3% @ p.2, 0% @ p.5 |
| CC9 | 95.6% @ p.2, 90.0% @ p.5 |
| Alk Phos | 12.2% @ p.2, 8.0% @ p.5 |
| CD45 | 0% @ p.2, 0.5% @ p.5 |
| Sterility: | Negative |
| Mycoplasma: | Negative |
| Endotoxin: | <0.05 EU/ml |

Cryopreserved smMPC-cyno and human MSC (huMPC) were thawed and seeded into differentiation assays optimised for human MPC differentiation along the chondrogenic, adipogenic and osteogenic pathways. Adipogenic differentiation and in vitro mineralisation were assessed by Oil-Red-O and Alizirin Red staining, respectively.

Like their huMPC counterparts, smMPC were capable of adipogenic differentiation (data not shown). Day 18 cultures of sm and huMPC were stained with Oil-Red-O for the presence of adipocytes. Both P1 and P5 cultures of smMPC harboured numerous lipid laden adipocytes when cultured in adipogenic culture conditions.

Following 21 days of osteogenic culture, cells were stained with alizarin red. Osteogenic differentiation was evidenced by the formation of red-staining mineral. Like huMPC, smMPC possess osteogenic potential.

### Laser-Induced Choroidal Neovasularization

Laser-induced choroidal neovascularization (CNV) was conducted on the same day as test article administration. The animals were food-deprived overnight prior to the procedure.

Prior to the procedure, mydriatic drops (1% mydriacyl) were applied to both eyes. The animals received an intramuscular injection of a sedative cocktail of glycopyrrolate, ketamine and xylazine, prior to anesthesia with isoflurane/oxygen. Under anesthesia, a 9-spot pattern was made around (not within) the macula of each eye using an 810 nm diode laser at an initial power setting of 250-300 mW and a duration of 0.1 seconds. In the event that rupture of Bruch's membrane is not confirmed for a particular spot an additional spot was added when considered appropriate by the veterinary ophthalmologist.

Hydration of the eyes was maintained with a saline solution during the procedure. Any notable events, such as retinal hemorrhage were documented for each laser spot.

### Administration of Test Article

Lucentis™ (0.5 mg/mL, 0.3 mL/vial; Novartis Canada) was administered at the time of laser treatment and the group receiving MPCs+Lucentis had MPCs administered 7 days after laser injury. Topical ophthalmic antibiotic (gentamicin) was applied to both eyes, twice on the day before treatment, immediately following the last injection and twice on the day following the injection (AM and PM). In cases where only one injection was performed prior to laser treatment, then the antibiotic was applied after the laser treatment.

The conjunctivae was be flushed with benzalkonium chloride (Zephiran™) diluted in Sterile Water, U.S.P. to 1:10,000 (v/v). A topical anesthetic (proparacaine, 0.5%) was applied to both eyes before and after the Zephiran™. A new syringe was used for each injection, using a 30-gauge, ½-inch needle. 50 μL of vehicle, test article cell suspension and/or Lucentis was administered bilaterally. Both eyes were examined immediately following treatment (indirect and/or direct ophthalmoscopy and/or slit-lamp biomicroscopy) to document any abnormalities caused by the injection procedure.

### Ocular Evaluations

### Ophthalmology

On Days 2, 14, 26, 34 and 40 animals were subjected to ophthalmology evaluations.

The mydriatic used was 1% mydriacyl. The animals were sedated for the examination. A sedative, Ketamine® HCl for Injection, U.S.P., was administered by intramuscular injection following an appropriate fasting period.

Examinations was performed by a board-certified veterinary ophthalmologist, first without mydriatic (slit lamp only) and then repeated following mydriatic administration (slit lamp and/or direct and/or indirect ophthalmoscopy). Fundic photographs of the eyes were taken for each animal pre-treatment, and as considered necessary by the veterinary ophthalmologist at ophthalmic examination.

### Tonometry

Intraocular pressure (IOP) was measured following the ophthalmic examinations (except for the immediate post dose examination). A local topical anesthetic (Alcain, 0.5%) was applied to the eyes prior to measurement. Measurements were made using a Tono-Pen XL™ or TonoVet. The same instrument type was used throughout the study.

### Electroretinography

Electroretinogram recordings were performed once pretreatment on all animals and on Days 27 and 41. Animals were dark adapted for at least 30 minutes prior to ERG recording. The animals received an intramuscular injection of a sedative cocktail of glycopyrrolate, ketamine and xylazine. Mydriacyl (1%) was applied to each eye approximately 5-10 minutes prior to the test. The eyelids were retracted by means of a lid speculum and a contact lens electrode placed on the surface of each eye. A needle electrode was placed cutaneously under each eye (reference) and on the head, posterior to the brow (ground). Carboxymethylcellulose (1%) drops were applied to the interior surface of the contact lens electrodes prior to placing them on the eyes.

Each ERG occasion consisted of the following series of scotopic single flash stimuli:
1) −30 dB single flash, average of 5 single flashes, 10 seconds between flashes
2) −10 dB single flash, average of 5 single flashes, 15 seconds between flashes.
3) 0 dB, average of 2 single flashes, approximately 120 seconds between flashes.

Following recording of the scotopic response, the animals were adapted to background light at approximately 25-30 cd/m2 for a period of approximately 5 minutes, followed by an average of 20 sweeps of photopic white flicker at 1 Hz, then 20 sweeps of photopic flicker at 29 Hz.

Fluorescein Angiography (excluding Group 7)

Fluorescein angiograms (FA) were obtained once predose and on Days 15, 28, 35 and 42. Following an appropriate fasting period, the animals received an intravenous injection of Propofol and then intubated.

Mydriacyl (1%) was applied to each eye approximately 5-10 minutes prior to the test. The eyelids were retracted by means of a lid speculum. Hydration of the eyes was maintained by frequent irrigation with saline solution. One mL of 10% sodium fluoresein was rapidly injected intravenously at which time the filling of the right eye were recorded for approximately 20 seconds in movie mode. Still images were recorded from both eyes approximately 2 and 10 minutes following fluorescein injection. The filling sequence was evaluated qualitatively. The individual laser spots on the still images were evaluated for leakage semiquantitively on a scale of 1-4.

### Statistical Analyses

Numerical data obtained during the conduct of the study from Groups 1 to 4 (Main Study only), were subjected to calculation of group mean values and standard deviations. For each parameter of interest (excluding ERG, tonometry and FA), group variances were compared using Levene's test at the 0.05 significance level. When differences between group variances were not found to be significant, a one-way analysis of variance (ANOVA) was performed. When significant differences among the means are indicated by the ANOVA (p≦0.05), then Dunnett's “t” test was used to perform the group mean comparisons between the control group and each treated group.

Whenever Levene's test indicated heterogeneous group variances (p≦0.05), the Kruskal-Wallis test was used to compare all considered groups. When the Kruskal-Wallis test was significant (p≦0.05), then the significance of the differences between the control group and each test group was assessed using Dunn's test. Data was evaluated on an individual basis and where appropriate group means and standard deviations were calculated.

For each pairwise group comparison of interest, significance was reported at the 0.05, 0.01 and 0.001 levels.

### Results

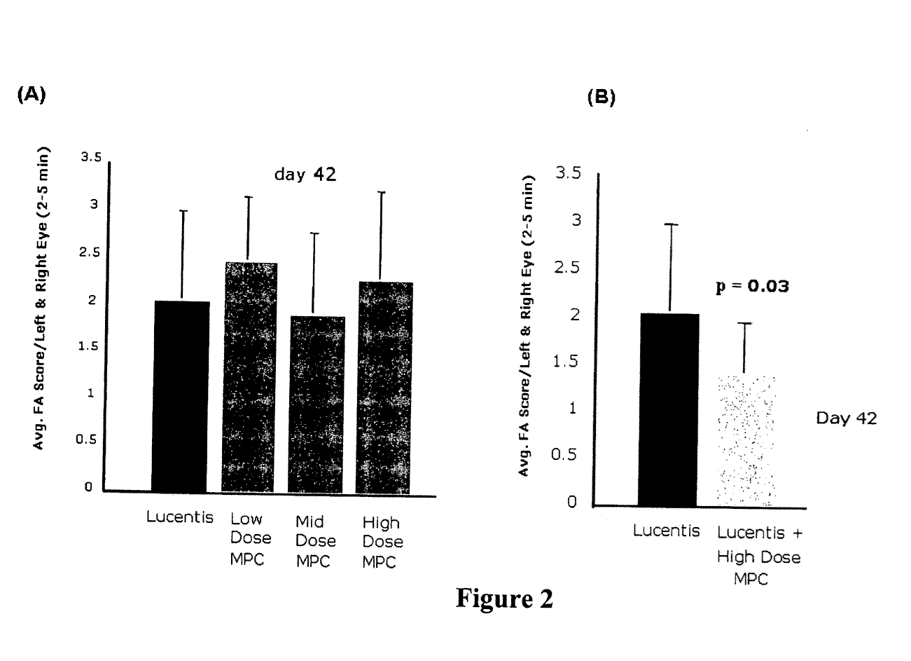

FIG. 2A shows the results of fluorescein angiography at day 42 after intravitreal injection of either anti-VEGF monoclonal antibody (Lucentis 0.5 mg/50 ul) or a single dose of allogeneic MPCs administered at low (78,100 cells/50 ul), medium (312,500 cells/50 ul), or high (1,250,000 cells/50 ul) concentration, injected in non-human primate eyes after laser photocoagulation. At day 42 post intravitreal injection, the degree of vessel leakage/neovascularization was comparable and not significantly different amongst any of the groups.

FIG. 2B shows that the additional injection of intravitreous allogeneic MPCs at the highest concentration (1,250,000 cells/50 ul) 7 days following intravitreal Lucentis (0.5 mg/50 ul) administration immediately post-laser photocoagulation resulted in a significantly reduced average fluorescein angiogram score at day 42, demonstrating a synergistic effect of the combination (p=0.03).

Fluorescein angiogram (FA) using 10% sodium fluoresein was rapidly injected intravenously with still images of each eye being captured approximately 2-5 minutes following administration. The angiograms were evaluated for leakage at day 42 using a semiquantitive grading scale of 1-4 for each spot that received laser photocoagulation.

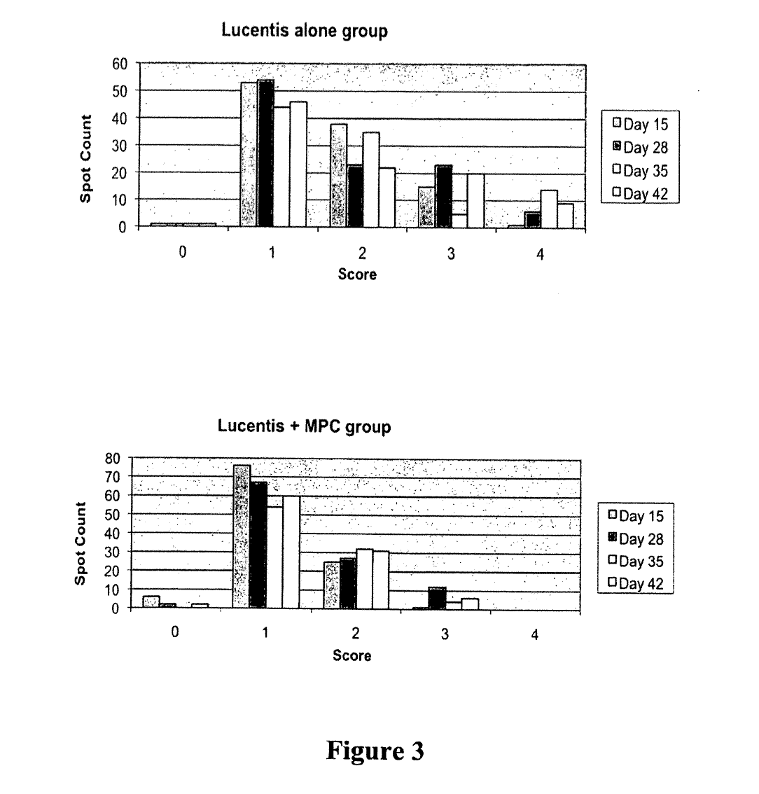

The combination treatment of allogeneic MPCs at the highest concentration (1,250,000 cells/50 ul) 7 days following Lucentis (0.5 mg/50 ul) administration immediately post-laser photocoagulation (lower panel) resulted in complete prevention of the most severe form of leaky vessels (grade 4 scoring) at all time points investigated (days 15, 28, 35 and 42), in contrast to many grade 4 severely leaky vessels being seen at all time points beyond day 15 in the Lucentis only group (FIG. 3).

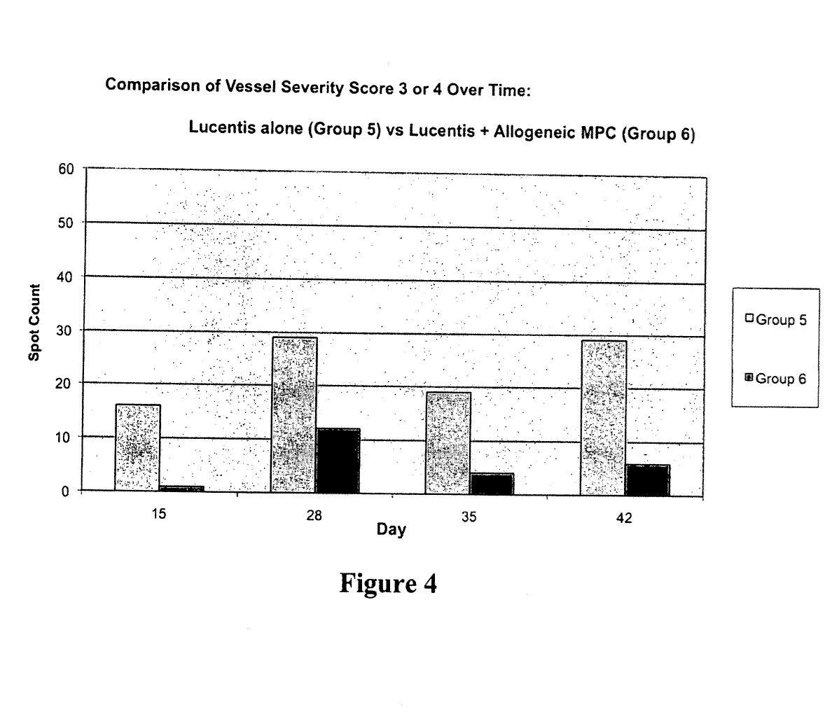

When all severe lesions were analysed (lesions of group 3 or 4 severity), the combination of allogeneic MPCs at the highest concentration (1,250,000 cells/50 ul) 7 days following Lucentis (0.5 mg/50 ul) administration immediately post-laser photocoagulation was shown to reduce severity of leaky vessels at all timepoints compared to Lucentis alone (FIG. 4). This effect was most significant at day 42 (p=0.013), at the study conclusion, indicating the long-term benefit of the synergistic effect.

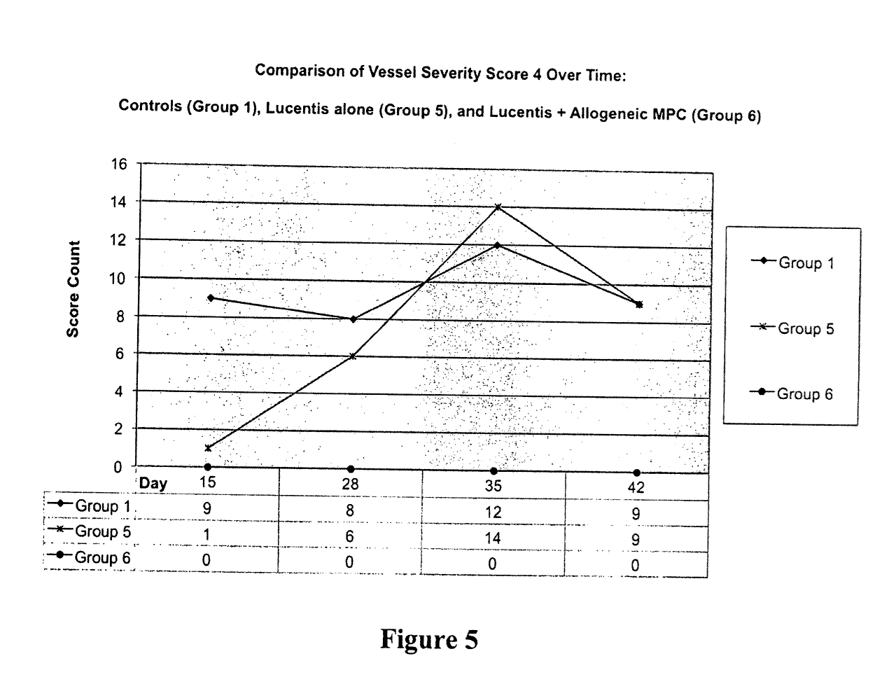

In comparison to controls receiving intravitreal injection of media alone, Lucentis treatment was found to be superior at day 15 in reducing grade 4 severe vessel leakage (FIG. 5). This effect was progressively lost beyond day 15, presumably reflecting the short half-life of the antibody. In contrast, combining Lucentis with allogeneic MPC at the highest dose at day 7 following laser coagulation injury completed prevented any grade 4 severe vessel lesions for the entire 42 day duration of the study. These results indicate that adding allogeneic MPC to Lucentis converted a transient effect of the anti-VEGF therapy on vessel leakage to a long-term, sustained effect.

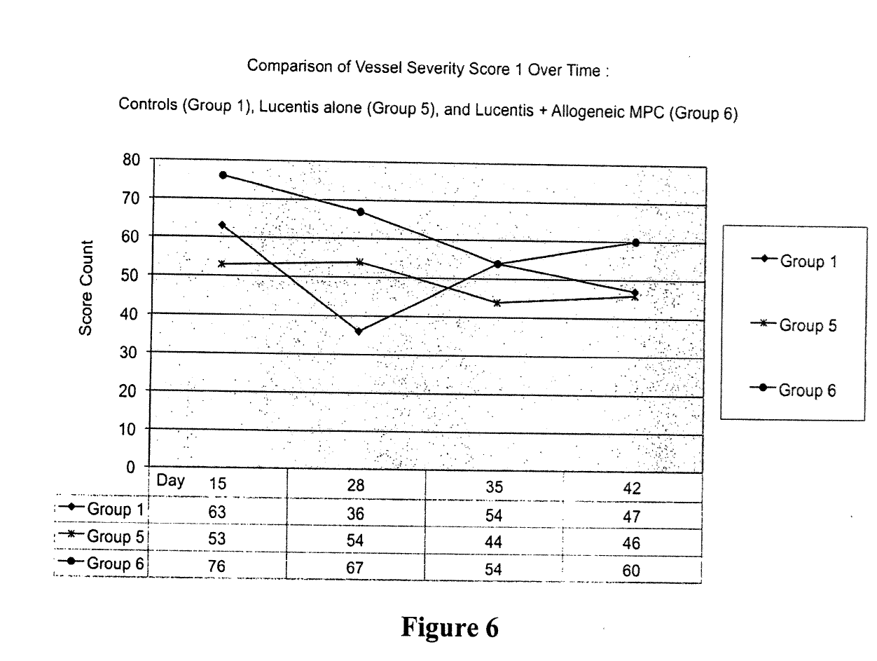

The combination of allogeneic high dose MPCs 7 days following Lucentis administration post-laser induced photocoagulation injury resulted in an increased number of low grade (grade 1) leaky vessels throughout the entire study period compared with either controls or animals receiving Lucentis alone (FIG. 6). This indicates that the combined therapy prevented progression of low severity vessels to high severity vessels, an effect that was seen early and was sustained throughout the period of study.

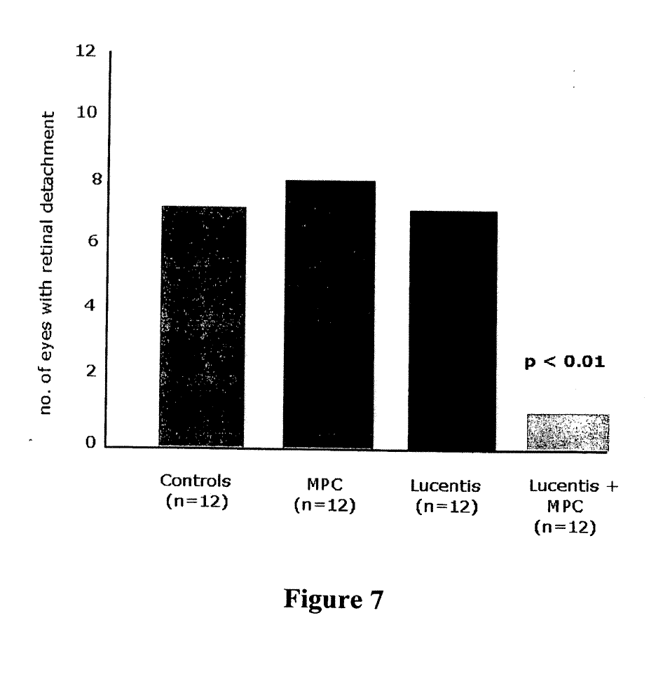

Histopathologic analysis at day 42 demonstrated that the combination therapy significantly reduced the incidence of retinal detachment compared to each of the other groups tested (p<0.01) (FIG. 7). Retinal detachment was seen in only 1/12 animals who received a combination of allogeneic MPCs at the highest concentration (1,250,000 cells/50 ul) 7 days following Lucentis (0.5 mg/50 ul) administration immediately post-laser photocoagulation. In contrast, retinal detachment was seen in 7/12 controls, 8/12 treated with high-dose MPC alone, and 7/12 treated with Lucentis alone.

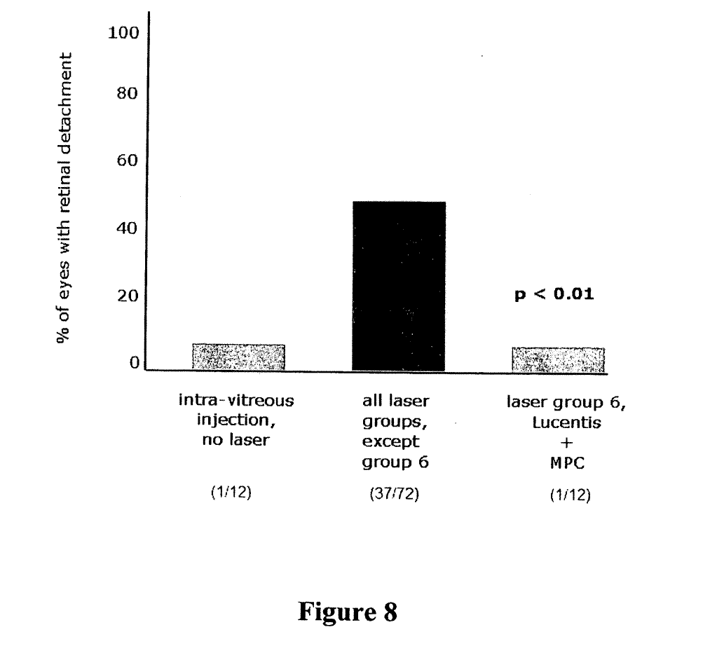

In comparison to the high incidence of retinal detachment among all animals receiving laser photocoagulation (37/72, 51%), only 1/12 animals (8.5%) who received a combination of allogeneic MPCs at the highest concentration (1,250,000 cells/50 ul) 7 days following Lucentis (0.5 mg/50 ul) administration immediately post-laser photocoagulation developed retinal detachment (p<0.01) (FIG. 8). These results indicate that the combination therapy had reduced the risk of retinal detachment associated with laser photocoagulation-induced neovascularization back to the baseline level seen with the intravitreous injection procedure alone.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

The present application claims priority from AU 2008903349 filed 30 Jun. 2008 and U.S. 61/133,607 filed 30 Jun. 2008, both of which are incorporated herein in their entirety by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Amarzguioui et al. (2004) Biochem Biophys Res Commun 316:1050-1058
Angiolillo et al. (1995) J Exp Med. 182: 155-162.
Bird et al (1988) Blood 80:1418-1422.
Breen (2007) J. Cell. Biochem. 102:1358-1367.
Cao et al. (1995) J Exp Med. 182: 2069-2077.
Chan et al. (2007) Retina 27:541-551.
Chau et al. (2005) Cancer Res. 65: 4918-4928.
Chen et al. (1995) Cancer Res. 55: 4230-4233.
Ciafrè et al. (2004) J. Vasc. Res. 41:220-228.
Clapp et al. (1993) Endocrinology. 133: 1292-1299.
Cross et al. (2003). Trends Biochem Sci. 28: 488-94.
Elbashire et al. (2001) Nature 411:494-498
Eremina and Quaggin (2004) Curr. Opin. Nephrol. Hypertens. 13:9-15.
Folkman (1971) N Engl J. Med. 285: 1182-1186.
Garg et al. (2008) Clin Experiment Ophthalmol 36: 252-256.
Good et al. (1990) Proc Natl Acad Sci USA. 87: 6624-6628.
Gronthos and Simmons. (1995) Blood 85: 929-940.
Gupta et al. (1995) Proc Natl Acad Sci USA. 92: 7799-7803.
Haseloff and Gerlach (1988) Nature 334:585-591.
Jones et al. (1986) Nature 321:522-525.
Kandel et al. (1991) Cell. 66: 1095-1104.
Kong et al. (2005) Cancer Res. 65: 9047-9055.
Kook et al. (2008) Ophthalmologe 105:158-164.
Kung et al. (2004) Cancer Cell 6: 33-43.
Huston et al. (1988) Proc Natl Acad Sci USA, 85:5879-5883.
Maione and Sharpe (1990) Trends Pharmacol Sci. 11: 457-461.
Miller (1990) Human Gene Therapy 1:5-14.
Morrison et al. (1984) Proc Natl Acad Sci USA 81:6851-6855.
Needleman and Wunsch. (1970). J. Mol. Biol., 48, 443-453.
O'Reilly et al. (1994) Cell. 79: 315-328.
Olofsson et al. (1996). Proc. Natl. Acad. Sci. USA. 93: 2576-2581.
Olsson et al. (2006). Nat. Rev. Mol. Cell. Biol. 7: 359-371.
Perriman et al. (1992) Gene 113: 157-16.
Rapisarda et al. (2002) Cancer Research 62: 4316-4324.
Reynolds et al. (2004) Nat. Biotech., 22:326-330
Senior (1998) Biotech. Genet. Engin. Revs. 15:79-119.
Shippy et al. (1999) Mol. Biotech. 12: 117-129.
Smith et al (2000) Nature 407: 319-320.
Strieter et al. (1995) Biophys Biochem Res Commun. 210: 51-57.
Su et al. (2007). Br. J. Cancer. 96: 541-545.
Sun et al. (1986) Hybridoma 5S1:517-520.
Tan et al. (2005) Cancer Res. 65: 605-612.
Tseng. et al. (2002) Surgery 132: 857-865.
Voest et al. (1995) J Natl Cancer Inst. 87: 581-586.
Waterhouse et al. (1998) Proc. Natl. Acad. Sci. USA 95: 13959-13964.
Weng et al. (2005) Mol. Cancer. Ther. 4: 948-955.
Yeo et al. (2003) J Natll Cancer Inst 95: 516-525.
Zhang et al. (2007) Ophthalmologica 222: 411-417.

1. A method of treating or preventing an eye disease or an angiogenesis-related disease, or both, in a subject, comprising administering to the subject i) cells, and ii) a compound that disrupts vascular endothelial growth factor (VEGF)-signalling.

2. A method of claim 1, wherein the eye disease is selected from the group consisting of: retinal ischemia, retinal inflammation, retinal edema, retinal detachment, macular hole, tractional retinopathy, vitreous hemorrhage, tractional maculopathy, diabetic retinopathy, diabetic macular edema, retinopathy of prematurity, macular degeneration, corneal graft rejection, neovascular glaucoma, retrolental fibroplasia and rubeosis.

3. A method of claim 1, wherein the eye disease is retinal detachment, diabetic retinopathy, retinopathy of prematurity or macular degeneration.

4. A method of claim 3, wherein the macular degeneration is dry age-related macular degeneration or wet age-related macular degeneration.

5. (canceled)

6. A method of claim 1, wherein the angiogenesis-related disease is selected from the group consisting of angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease and Helicobacter pylori ulcers.

7. A method of claim 1, wherein the cells are stem cells, or progeny cells thereof.

8. A method of claim 7, wherein the stem cells are obtained from bone marrow or the eye.

9. A method of claim 7, wherein the stem cells are mesenchymal precursor cells (MPC).

10. A method of claim 9, wherein the mesenchymal precursor cells are TNAP⁺, STRO-1⁺, VCAM-1⁺, STRO-2⁺, CD45⁺, CD146⁺, or 3G5⁺ or any combination thereof.

11. A method of claim 10, wherein at least some of the STRO-1⁺ cells are STRO-1^{bri}.

12. A method of claim 9, wherein the progeny cells are obtained by culturing mesenchymal precursor cells in vitro.

13. A method claim 1, wherein the compound binds, or reduces the production of, or both binds and reduces the production of, a vascular endothelial growth factor.

14. (canceled)

15. (canceled)

16. A method of claim 13, wherein the vascular endothelial is hypoxia-inducible factor 1 (HIF-1).

17. A method of claim 1, wherein the compound binds or reduces the production of, or both binds and reduces the production of, a vascular endothelial growth factor receptor.

18. (canceled)

19. (canceled)

20. A method of claim 1, wherein the compound binds or reduces the production of, or both binds and reduces the production of, a molecule involved in intracellular signalling induced by a vascular endothelial growth factor binding a vascular endothelial growth factor receptor.

21. A method of claim 1, wherein the compound is a polypeptide or a polynucleotide.

22. A method of claim 21, wherein the polypeptide is an antibody, an antibody-related molecule, and/or a fragment of an antibody or an antibody-related molecule; or the polynucleotide is, or encodes, an antisense polynucleotide, a sense polynucleotide, a catalytic polynucleotide, or a duplex RNA molecule.

23. (canceled)

24. (canceled)

25. A method of claim 1, wherein at least some of the cells are genetically modified.

26. (canceled)

27. (canceled)

28. (canceled)

29. (canceled)

30. A composition comprising cells and a compound that disrupts VEGF-signalling.

31. (canceled)
